SEQUENCE LISTING (18 sequences)

SEQ ID NO: 1 - DNA, 20 bp, Artificial sequence
  FXR (Nr1h4) - Forward
gctctgctcacagcgatcgt

SEQ ID NO: 2 - DNA, 35 bp, Artificial sequence
  FXR (Nr1h4) - Probe
atcctctctccagacagacaatacatcaaggacag

SEQ ID NO: 3 - DNA, 20 bp, Artificial sequence
  FXR (Nr1h4) - Reverse
ctcctgcagcttctccaccg

SEQ ID NO: 4 - DNA, 21 bp, Artificial sequence
  Shp-1 (NrOb2) - Forward
tgaagggcacgatcctcttca

SEQ ID NO: 5 - DNA, 20 bp, Artificial sequence
  Shp-1 (NrOb2) - Probe
atgtgccaggcctccgtgcc

SEQ ID NO: 6 - DNA, 19 bp, Artificial sequence
  Shp-1 (NrOb2) - Reverse
caggtgtgcgatgtggcag

SEQ ID NO: 7 - DNA, 28 bp, Artificial sequence
  IBABP (Fabp6) - Forward
ttgagagtgagaagaattacgatgagtt

SEQ ID NO: 8 - DNA, 23 bp, Artificial sequence
  IBABP (Fabp6) - Probe
atgaagcgcctgggtcttccagg

SEQ ID NO: 9 - DNA, 20 bp, Artificial sequence
  IBABP (Fabp6) - Reverse
cgtcccctttcaatcacgtc

SEQ ID NO: 10 - DNA, 22 bp, Artificial sequence
  IBABP (Fabp6)) - Forward
gggagtattctgactgcgttgc

SEQ ID NO: 11 - DNA, 35 bp, Artificial sequence
  IBABP (Fabp6) - Probe
catttcttcatgggatattacaggtatgcaacaga

SEQ ID NO: 12 - DNA, 26 bp, Artificial sequence
  IBABP (Fabp6) - Reverse
tgactaaacaggtcaacgtggagtta

SEQ ID NO: 13 - DNA, 20 bp, Artificial sequence
  VDR (Nr1i1) - Forward
ccatctgcattgtctcccca

SEQ ID NO: 14 - DNA, 27 bp, Artificial sequence
  VDR (Nr1i1) - Probe
agccattcaggaccgcctatccaacac

SEQ ID NO: 15 - DNA, 19 bp, Artificial sequence
  VDR (Nr1i1) - Reverse
gcagcggatgtaggtctgc

SEQ ID NO: 16 - DNA, 21 bp, Artificial sequence
  PXR (Nr1i2) - Forward
tggccgatgtgtcaacctaca

SEQ ID NO: 17 - DNA, 32 bp, Artificial sequence
  PXR (Nr1i2) - Probe
cgtcatcaacttcgccaaagtcatatcctact

SEQ ID NO: 18 - DNA, 19 bp, Artificial sequence
  PXR (Nr1i2) - Reverse
aaaagtggcccccttcagc

(19) United States
(12) Patent Application Publication
(10) Pub. No.: US 2009/0010923 A1
(43) Pub. Date: Jan. 8, 2009

(54) TREATMENT OF CANCER WITH ANTI-MUSCARINIC RECEPTOR AGENTS

(75) Inventors: Jean-Pierre Raufman, Baltimore, MD (US); Kunrong Cheng, Cockeysville, MD (US); Jasleen Shant, Elkridge, MD (US); Guofeng Xie, Baltimore, MD (US); Sandeep Khurana, Columbia, MD (US)

Correspondence Address: MORGAN LEWIS & BOCKIUS LLP, 1111 PENNSYLVANIA AVENUE NW, WASHINGTON, DC, 20004, US

(73) Assignee: University of Maryland, Baltimore, Baltimore, MD (US)

(21) Appl. No.: 12/109,156

(22) Filed: Apr. 24, 2008

Related U.S. Application Data
(60) Provisional application No. 60/913,800, filed on Apr. 24, 2007.

(51) Int. Cl. A61K 39/395; A61P 35/00; A61K 31/7088; A01K 67/027; A61K 31/713

(52) U.S. Cl. 424/130.1; 514/44; 800/18

(57) ABSTRACT

The present invention relates to methods of treating proliferative disorders of colon cells. The methods comprise administering to the cells an effective amount of at least one agent to reduce M3 muscarinic receptor-mediated transactivation of at least one epidermal growth factor receptor.

### STATE REGARDING FEDERALLY SPONSORED RESEARCH OR DEVELOPMENT

Part of the work performed during development of this invention utilized U.S. Government funds, through the National Institutes of Health Grant Nos. CA107345 and DK067872, as well as funds through Research Service, Department of Veterans Affairs. The U.S. Government has certain rights in this invention.

### BACKGROUND OF THE INVENTION

1. Field of the Invention

The present invention relates to methods of treating cancer by reducing or inhibiting tumor proliferation in subjects in need of treatment with anti-muscarinic agents.

2. Background of the Invention

There are several different types of acetylcholine (ACh) receptors present on different organs and tissues in the body. These ACh receptors are generally divided into “muscarinic” and “nicotinic” classes of ACh receptors. Cholinergic receptors, which bind to and are activated by the alkaloid, muscarine, are called muscarinic receptors.

The muscarinic receptors are G-protein coupled receptors (GPCRs) that are widely distributed on multiple organs and tissues, and are critical to the maintenance of central and peripheral cholinergic neurotransmission. The muscarinic class is further subdivided into M1, M2, M3, M4, and M5 subclasses. (Bonner et al., Science, 237 : 527-532, 1987); Brann et al., Prog, Brain Res., 98 : 121-127, 1993). The activity of the M1, M3, and M5 receptors are mediated by the phosphoinositide second-messenger system, and the M2 and M4 receptors are linked to the adenylate cyclase second messenger system. (Bonner et al. 1987). The regional distribution of these receptor sub-types in the brain and other organs has been documented. For example, the muscarinic M₃R receptors is present in glands and smooth muscle, and the M₃R receptor mediates the excitatory effects of the parasympathetic system on glandular secretion and on the contraction of visceral smooth muscle. Chapter 6, Cholinergic Transmission, in H. P. Rang et al., Pharmacology, Churchill Livingstone, New York (1995).

Cancer cells have been found to express muscarinic M3 receptors, e.g., H508 colon cancer cells. Frucht et al., Cancer Res., 52: 1114-1122, 1992 ; Frucht et al., Clin. Cancer Res., 5: 2532-2539, 1999. Activation of muscarinic M3 receptors have been found to stimulate H508 cell proliferation. Frucht et al., 1999. In particular, the activation of muscarinic receptors has been reported to transactivate EGFR, resulting in phosphorylation of the EFGR signaling cascade. Slack et al., Biochem. J., 348; 381-387, 2000.

M₃R, which is also expressed widely in the gastrointestinal tract, couples to G_{q11} and activates phospholipase C signaling and inositol phosphate (IP) formation, thereby increasing cell calcium. A cell line derived from a moderately differentiated cecal adenocarcinoma designated NCI-H508 (shortened to H508 in this application) has a particularly high level of M₃R expression (approx. 2500 binding sites/cell). H508 cells express only the M₃R. subtype. Previously, M₃R was shown to be expressed in both normal and cancerous colon tissue and there was up to an 8-fold increase in M₃R expression in cancer tissue compared to normal tissue. Collectively, these findings indicate the importance of M₃R expression for colon carcinogenesis.

In H508 colon cancer cells, acetylcholine (ACh) activates p44/42 MAPK and p90RSK, which is a nuclear response protein that regulates gene expression and the cell cycle. The present inventors examined the actions of ACh in H508 cells, CHO cells expressing rat M₃R (CHO-rM₃R), and SNU-C4 colon cancer cells that do not express muscarinic receptors. ACh induced p44/42 MAPK phosphorylation in H508 and CHO-rM₃R cells (14- and 27-fold, respectively), but not in SNU-C4 cells. After adding a calcium chelator, an inhibitor of MEK (MAPK kinase; the regulatory protein just upstream of p44/42 MAPK) and atropine inhibited the actions of ACh. These findings in human colon cancer cells show that proliferative actions of muscarinic agonists are dependent on M₃R expression and activation of post-M₃R signaling.

Epidermal growth factor receptor (EGFR) is a member of the growth factor receptor family, and is characterized by a single membrane-spanning domain and intrinsic tyrosine kinase activity. Cheng et al., Cancer Res., 63: 6744-6750,2003. The cellular mechanism underlying cholinergic agonist-induced H508 cell proliferation has been found to be mediated by transactivation of EGFR. Cheng et al., 2003.

In cancer, EGFR transactivation by GPCR's (e.g. M₃R) is a common feature of mitogenic signaling. Presently it has been demonstrated that EGFR inhibitors reduced ACh-induced MAPK phosphorylation in H508 cells, but not in CHO-rM₃R cells negative for EGFR. In H508cells, incubation with ACh caused robust phosphorylation of EGFR Tyr⁹⁹². EGFR Tyr⁹⁹² phosphorylation is important for PLCα activation and subsequent calcium signaling. EGFR inhibitors blocked ACh- and EGF-induced Tyr⁹⁹² phosphorylation and cell proliferation. These findings indicate that the actions of ACh are mediated by transactivation of EGFR in colon cancer cells.

Colon cancer is the second most common cause of cancer death and the leading gastrointestinal cause of death. Experimental data support the concept that colon cancers arise as a consequence of progression from normal colonic mucosa to adenomatous polyp to cancer, associated with accumulation of somatic genetic alterations that affect the regulation of apoptosis and DNA repair. These alterations include mutations and/or methylation of oncogenes, tumor suppressor and mismatch repair genes. Environmental factors (e.g. fecal bile acids) also play an important role in colon carcinogenesis. Population studies associate colorectal cancer with elevated fecal bile acid concentration, particularly lithocholic and deoxycholic acids. Experiments using rodents treated with carcinogens indicate that direct instillation or other means of increasing fecal bile acids augments development of colon cancer. In rats, feeding secondary bile acids increases the risk of colon epithelial dysplasia and reducing fecal deoxycholic acid with oral ursodeoxycholic acid (ursodiol) decreases colon tumor formation. In humans with inflammatory bowel disease, modulating fecal bile acids with oral ursodiol reduces the incidence of both colon epithelial dysplasia and cancer.

However, a defined mechanism in which bile acids promote colon cancer is unknown. Studies by Martinez and colleagues showing that deoxycholic acid induces apoptosis in colon cancer cell lines do not explain the tumor-promoting properties of bile acids. Later studies indicate that deoxycholic acid alters MAP kinase signaling in colon cancer cells, thereby suppressing p53 activity. Although EGFR activation was implicated in this process, the signaling cascades responsible for these actions were not elucidated and the role of bile acid interactions with plasma membrane or nuclear receptors was not explored.

Most human colon cancers over-express M₃R and cholinergic agonists stimulate colon cancer cell proliferation. The present inventors have unexpectedly found that that cholinergic agonists, including bile acids, interact with M₃R on colon cancers cells, thereby causing transactivation of EGFR and stimulating proliferation, indicating a likely molecular mechanism underlying carcinogenic actions of bile acids.

The present inventors have found unexpected results that lithocholyltaurine (LCT), a lithocholic acid conjugate, interacts with muscarinic receptors on gastric chief cells, thereby stimulating pepsinogen secretion. Evidence for the specific and functional nature of this interaction included the findings that atropine inhibited the actions of LCT, that LCT competed for binding with a muscarinic radioligand, and that LCT increased IP formation. In contrast to LCT, cholyltaurine and taurine controls did not alter these parameters. In addition, lithocholic and deoxycholic acid conjugates interact selectively and functionally with CHO cells that express each of the 5 muscarinic receptor subtypes.

In a systematic analysis of bile acid interaction with CHO cells expressing muscarinic receptor subtypes, deoxycholic acid conjugates are observed to inhibit binding of N-methylscopolamine, which is a muscarinic radioligand, to cells expressing M₃R. Deoxycholic acid conjugates are more abundant in the gut and more soluble than lithocholic acid conjugates.

Because muscarinic effects on colon cancer cell proliferation are mediated by transactivation of EGFR, results of bile acid interaction with M₃R depend on the cell type examined. In CHO cells that express M₃R but not EGFR, deoxycholic acid conjugates are muscarinic receptor antagonists. In contrast, in H508 and HT-29 colon cancer cells that express both M₃R and EGFR, bile acids stimulate EGFR signaling and cell proliferation. Thus, in cells co-expressing M₃R and EGFR deoxycholic acid conjugates are muscarinic receptor agonists whose effects are mediated by transactivation of EGFR.

Bile acids are products of cholesterol metabolism. In humans, primary bile acids (cholic and chenodeoxycholic acids) are synthesized in the liver and conjugated with glycine or taurine. Conjugated bile acids undergo active reabsorption in the distal ileum and return to the liver (enterohepatic circulation). In the distal small intestine and proximal colon bacterial enzymes deconjugate bile acids. Most are absorbed passively and return to the liver for re-conjugation. Also in the small intestine and colon, bacterial 7α-dehydroxylation of cholic and chenodeoxycholic acids results in formation of secondary bile acids (deoxycholic and lithocholic acids, respectively). Cholic, chenodeoxycholic, deoxycholic and lithocholic acids comprise >95% of the human bile acid pool. Bile acids stimulate human colon cancer cell proliferation by muscarinic mechanisms.

It is important to demonstrate in vivo that bile acids achieve concentrations necessary for interaction with M₃R. Because of limited access, however, it is difficult to measure bile acid concentrations in human intestinal compartments. Because of species differences in bile acid composition, experimental data in animals cannot be applied to humans. In humans, reported concentrations for total bile acids are 2 to 45 mM in bile; 10 mM in jejunum and 1 mM in the cecum. Lithocholic and deoxycholic acid derivatives in the proximal colon are reported in the high mieromolar to millimolar range, particularly if ileal damage reduces enterohepatic circulation.

Maximal effects of bile acids on signaling and proliferation in colon cancer cells are of lower magnitude than observed with ACh. Nonetheless, several factors argue that bile acids may play an important role for the following reasons: (a) fecal bile acids are in contact with colonic epithelium for many years (the average age for developing colon cancer is greater than 50 years); (b) bile acids lack an ester linkage and are not hydrolyzed by tissue cholinesterases that rapidly inactivate ACh; (c) lipophilic lithocholic acid derivatives have access to muscarinic receptors in the lipid bilayer of colon cancer cell membranes (e.g. the novel bile acid: ACh hybrid molecule, lithocholylcholine, interacts with muscarinic receptors on rat aortic strips); (d) neoplastic cells commonly lose cell membrane polarity, thereby leading to expression on the apical membrane of receptors usually restricted to the basolateral membrane; and (e) colon cancers have increased tight junction permeability, thereby increasing access of luminal molecules to basolateral membrane receptors.

Epidemiological and animal studies associate colon cancer risk with alterations in the spectrum and concentration of fecal bile acids. Published data indicate that bile acids interact functionally with M₃R expressed on human colon cancer cells and that interaction of cholinergic agonists, including bile acids, with M₃R results in transactivation of epidermal growth factor receptors (EGFR).

The significance of the present invention is that in the United States, for both men and women colon cancer is a leading cause of morbidity and mortality. Colon cancer screening reduces death rates, but efficacy is limited by patient non-compliance, ‘miss’ rates on colonoscopy, and other factors. Chemoprevention using eyclooxygenase-2 inhibitors is effective, but benefits are limited by toxicity. Cardiotoxicity resulted in withdrawal of one agent (rofecoxib) from the market. Efficacy of non-surgical treatment (e.g. chemotherapy and radiation) for advanced colon cancer is limited. Because approximately one-third of persons diagnosed with colon cancer die from this disease, new approaches are needed. Because the present inventors have found that conjugated bile acids stimulate proliferation of colon cancer cells that express M₃R and that azoxymethane (AOM)-treated M₃R-deficient mice have a dramatic reduction in tumor formation, M₃R has now been shown to have a novel role in promoting colon carcinogenesis. M₃R is an excellent therapeutic target and will reveal a novel, low-risk strategy for both preventing and treating this disease.

Thus, in light of the role of muscarinic M3 receptors in cholinergic-induced proliferation of cancer cells, anti-muscarinic agents will allow detection, treatment, and prevention of tumor proliferation and metastases by targeting the muscarinic M3 receptor to reduce and/or inhibit muscarinic M3 receptor-mediated transactivation of EGFR.

In addition, the present inventors have found that in murine models of colon cancer, M₃R expression and activation is important for bile acid-induced intestinal tumor formation and that M₃R deficiency greatly reduces the number of colon tumors observed in AOM-treated mice. A novel strain of Apc^{Min/+} mice with variable expression of M₃R is produced in the present invention. This animal colon cancer model may be used to establish the importance of M₃R expression for development of colonic neoplasia, to identify molecular mechanisms whereby bile acid-induced activation of M₃R promotes carcinogenesis, to determine the role of bile acid nuclear receptors in mediating these actions, and to determine whether pharmacological inhibition of M₃R activation mimics M₃R gene ablation.

### SUMMARY OF THE INVENTION

The present invention relates to methods for reducing tumor proliferation or tumor metastasis in a subject comprising administering to a subject in need of treatment a therapeutically effective amount of at least one agent to reduce M3 muscarinic receptor activity. In one embodiment, the receptor activity is transactivation of at least one epidermal growth factor receptor.

The present invention relates to methods for reducing cell proliferation in a subject comprising administering to a subject in need of treatment a therapeutically effective amount of at least one agent to reduce M3 muscarinic receptor-mediated transactivation of at least one epidermal growth factor receptor.

The present invention also relates to methods for inhibiting tumor proliferation in a subject comprising administering to a subject in need of treatment a therapeutically effective amount of at least one agent to inhibit M3 muscarinic receptor expression.

The present invention also relates to methods for inhibiting cell proliferation in a subject comprising administering to a subject in need of treatment a therapeutically effective amount of at least one agent to inhibit M3 muscarinic receptor expression.

The present invention relates to methods for reducing tumor metastasis in a subject comprising administering to a subject in need of treatment a therapeutically effective amount of at least one agent to reduce M3 muscarinic receptor-mediated transactivation of at least one epidermal growth factor receptor.

The present invention also relates to methods for inhibiting tumor metastasis in a subject comprising administering to a subject in need of treatment a therapeutically effective amount of at least one agent to inhibit M3 muscarinic receptor expression.

Another aspect of the present invention is related to a method of reducing the likelihood of tumor development in a subject comprising administering to a subject in need of treatment a therapeutically effective amount of at least one agent to reduce muscarinic receptor-mediated transactivation of at least one epidermal growth factor receptor.

The present invention also relates to transgenic animals whose genome encodes a transgene comprising a mutated murine muscarinic M₃ receptor gene and mutated adenomatous polyposis coli gene, wherein the mutated murine muscarinic M₃ receptor gene results in a reduction in muscarinic M₃ receptor gene expression and at least about 50% reduction in tumor proliferation in the colon compared to a wild-type mouse.

### BRIEF DESCRIPTION OF THE DRAWINGS

FIG. 1 depicts bile acid-induced colon cancer cell proliferation which requires M₃R expression and activation in H508 and SNU-C4 cells. H508 (A,C) and SNU-C4 cells (B) were treated with ACh, LCT, DCG, or DCT (5 days, 37° C.) alone (A,B) or with atropine (C). Cell proliferation was determined using the sulforhodamine B (SRB) colorimetric assay. Mean±SEM of 3 exp. (A): *,** significantly greater than water (p<0.05 and 0.001, respectively, unpaired Student's Mest). (C): significantly less than DCT or ACh alone (p<0.05 and 0.001, respectively). ACh, lithocholic and deoxycholic acid conjugates stimulate proliferation of H508 cells that co-express M₃R and EGFR, but do not significantly alter proliferation of SNU-C4 cells that express EGFR but not M₃R. As observed, actions of DCT are inhibited by atropine.

FIG. 2 depicts markers of cell proliferation and apoptosis in colon epithelial cells from PBS- and AOM-treated WT and M3R−/− mice. A, representative BrdU-stained sections from PBS-treated WT, AOM-treated WT, and AOM-treated M3R−/− mice. Arrows indicate representative BrdU-stained nuclei. B, percentage of BrdU-positive cells in colon epithelial cells in the three treatment groups. Columns, mean percent BrdU-stained cells; bars, SE. N=5 animals/group. Unpaired Student's t-test. C, representative activated caspase-3-stamed sections from PBS-treated WT, AOM-treated WT, and AOM-treated M3R−/− mice. Arrows indicate activated caspase-3-stained cells. D, percentage of activated caspase-3-positive cells in colon epithelial cells in the three treatment groups. Columns, mean percent activated caspase-3-stained cells; bars, SE, N=5 animals/group. Unpaired Student's t-test.

FIG. 3 depicts ACF and BCAC staining. Identification of aberrant crypt foci (ACF) and β-catenin-accumulating crypt (BCAC). FIG. 3A and B shows an example of methylene blue-stained ACF in colon resected from AOM-treated WT mouse. This ACF, shown at low-(A) and high-power magnification (B), has >20 aberrant crypts. FIG. 3C shows BCAC stained by immunohistochemistry. ACF and BCAC staining allow identification of early markers of epithelial neoplasia. Formalin-fixed colon from AOM-treated WT mouse was used to detect ACF (0.25% methylene blue) (A+B) and BCAC (C+D). A+B: ACF (arrows) at X40 and X100 original magnification, respectively. C: BCAC (arrows), X100.

FIG. 4 depicts macroscopic colon tumors in PBS- and AOM-treated WT and M3R−/− mice. A, representative dissecting microscope images of colon mucosa from PBS-treated WT (top), AOM-treated WT (middle) and AOM-treated M3R−/− mice (bottom). Macroscopic tumors >0.5 mm diameter indicated by dashed circles, respectively. Number of tumors/section indicated at bottom of each image, size bars, 1 mm. B, reduced tumor number in M3R−/− compared to WT mice treated with AOM (mean±SE; P<0.05, Mann-Whitney U test). N, number of mice/treatment group. C, reduced tumor volume in M3R−/− compared to WT mice (mean±SE, P<0.05, Mann-Whitney U test). Columns, mean tumor number per mouse colon; bars, SE. N, number of mice/treatment group.

FIG. 5 AOM-induced tumors mimic human adenomas and adenocarcinomas, and are reduced in M₃R-deficient mice. A, photograph of two representative adenocarcinomas in the distal colon of an AOM-treated WT mouse. B, representative hematoxylin and eosin-stained microscopic images of adenoma and adenocarcinoma in AOM-treated WT mice. Left, adenoma (X20 magnification). Middle, poorly differentiated adenocarcinoma (X20 magnification). Right, higher magnification of indicated area of adenocarcinoma (X40 magnification). * indicates luminal surface. Green arrowheads delineate muscularis mucosa. Black arrows indicate poorly formed glands. C, number of adenomas and adenocarcinomas per mouse colon are reduced in M3R-deticient mice. Columns, mean number of adenomas and adenocarcinomas per mouse colon; bars, SE. N=number of mice/treatment group. Unpaired Student's t-test.

FIG. 6 depicts WT and M3R−/− mouse colon mucosal histology and muscarinic receptor expression. A, representative hematoxylin and eosin-stained colon sections from WT and M3R−/− mice. B, in situ hybridization using specific M1R and M3R riboprobes in colon epithelium from WT and M3R−/− mice.

FIG. 7 depicts reduction in colon tumor number in Apc^{Min/+} mice that are negative for the M3R receptor.

### DETAILED DESCRIPTION OF THE INVENTION

The present invention relates to methods of treating proliferative disorders of colon cells. The methods comprise administering to the cells an effective amount of at least one agent to reduce M3 muscarinic receptor-mediated transactivation of at least one epidermal growth factor receptor.

As used herein, “receptor” refers to a macromolecular binding site (usually a protein, which may also be glycosylated or phosphorylated) which is at least partially exposed on the surface of a cell, and which has specific and limited affinity for one or more fluid-borne molecules, called “ligands” (these usually, but not necessarily, are neurotransmitters or hormones). A cell or cell, surface need not be present, provided the macromolecular binding site is available to bind a ligand.

When a ligand contacts an appropriate receptor, a brief binding reaction occurs which causes a cellular response, such as opening of an ion channel, which can lead to activation and depolarization of the neuron. Most receptor molecules are proteins that straddle the membrane of a cell, with an external portion for binding reactions, and an internal portion that can trigger cellular responses or cascades that occurs when the receptor is activated by a ligand.

The agents used in accordance with the present invention includes, but are not limited to, muscarinic receptor antagonist, anti-muscarinic receptor antibody, anti-muscarinic receptor nucleic acid sequences, ribozymes, or a combination thereof.

The agent may be combined with other antineoplastic agents including, but not limited to, alkylating agents (e.g., cyclophosphamide, ifosfamide, thiotepa, melphalan, busulfan, carmustine, cisplatin, carboplatin), antimetabolites (e.g., ,methotrexate, fluorodeoxyuridine, cytarabine, mercaptopurine, azathiopurine, thioguanine, fludarabine phosphate, pentostatin, cladribine), natural products (e.g., vinblastine, vincristine, vinorelbine, paclitaxel, etoposide, teniposide, dactinomycin, daunorubicin, doxorubicin, idarubicin, bleomycins, plicamycin, mitomycin, L-asparaginase), hormones and related agents (e.g., adrenocorticosteroids, aminoglutethimide, progestins, estrogens and androgens, antiestrogens, gonadotropin-releasing hormone analogs, interleukin-2), or a combination thereof.

Pharmacologically, an “agonist” is a molecule that activates a receptor. In contrast, an “antagonist” is a molecule that prevents or reduces the effects exerted by an agonist at a receptor. “Muscarinic antagonists” useful in the methods of the present invention are agents that act to inhibit one or more characteristic responses of a muscarinic receptor, for example, by competitively or non-competitively binding to the muscarinic receptor, by binding to a ligand of the receptor and/or to a downstream signaling molecule.

Compounds having antagonistic activity against muscarinic receptors have been described in the following references, all of which are herein incorporated by reference: Japanese patent application Laid Open Number 92921/1994 and 135958/1994; WO 93/16048; U.S. Pat. No. 3,176,019; GB 940,540; EP 0325 571; WO 98/29402; EP 0801067; EP 0388054; WO 9109013; U.S. Pat. No. 5,281,601. Also, U.S. Pat. Nos. 6,174,900, 6,130,232 and 5,948,792; WO 97/45414 are related to 1,4-disubstituted piperidine derivatives; WO 98/05641 describes fluorinated, 1,4-disubstituted piperidine derivatives; WO 93/16018 and W096/33973. U.S. Pat. No. 5,397,800 discloses l-azabicyclo[2.2.1]heptanes. U.S. Pat. No. 5,001,160 describes 1-aryl-1-hydroxy-1-substituted-3-(4-substituted-1-piperazinyl)-2-propanon- WO 01/42213 describes 2-biphenyl-4-piperidinyl ureas. WO 01/42212 describes carbamate derivatives. WO 01/90081describes amino alkyl lactam. WO 02/53564 describes novel quinuclidine derivatives. WO 02/00652 describes carbamates derived from arylaikyl amines. WO 02/06241 describes 1,2,3,5-tetrahydrobenzo(c)azepin-4-one derivatives.

Specific examples of muscarinic M₃ receptor antagonists that may be used in accordance with the present invention include, but are not limited to, 4-[(diphenylacetyl)oxy]-1,1-dimethylpiperidinium iodide (4-DAMP), ipratropium bromide, tiotropium bromide, oxitropium bromide, perenzepine, telenzepine, darifenacin, or a combination thereof. Agents that may also be used in the methods of the present invention include M3 selective antagonists such as the cyclohexylmethyl piperidinyl triphenylpropioamide derivatives described in J. Med. Chem., 44, p, 984 (2002), or the quinuclidine carbamate derivatives described in U.S. Application No. 20040266816, which are both herein incorporated by reference.

One aspect of the present invention relates to methods of inhibiting colon tumor cell proliferation or tumor metastasis by inhibiting muscarinic M3 receptor expression by administering agents including, but not limited to, muscarinic M3 receptor antibodies, anti-M3muscarinic receptor nucleic acid sequences, ribozymes, or a combination thereof.

The anti-M3 muscarinic receptor antibody of the present invention is directed against the muscarinic M₃ receptor and may be used as a tumor marker and/or immunotherapy target for tissues such as glands and smooth muscle, in which the muscarinic M3 receptor is expressed. The polynucleotide sequences of the muscarinic M3 receptor are publicly available and accessible through sequence databases, and the anti-M3 muscarinic receptor antibodies of the present invention may be made according to a variety of methods well-known in the art.

Cells expressing the polypeptide of the muscarinic M3 receptor of the present invention are administered to an animal to induce the production of sera containing polyclonal antibodies. In a preferred method, a preparation of a polypeptide of the muscarinic M3 receptor of the present invention is prepared and purified to render it substantially free of natural contaminants. Such a preparation is then introduced into an animal in order to produce polyclonal antisera of greater specific activity.

Monoclonal antibodies specific for muscarinic M3 receptors of the present invention are prepared using hybridoma technology. (Kohler et al., Nature 256:495 (1975); Kohler et al., Eur. J. Immunol. 6:511 (1976); Kohler et al., Eur. J. Immunol. 6:292 (1976); Hammerlinget al, in: Monoclonal Antibodies and T-Ce!l Hybridomas, Elsevier, N.Y., pp. 563-681 (1981), which are all herein incorporated by reference). Generally, an animal is immunized with a polypeptide of the muscarinic M3 receptor of the present invention, or with a secreted muscarinic M3 receptor polypeptide-expressing cell. Such muscarinic M3 receptor polypeptide-expressing cells are cultured in any suitable tissue culture medium, such as Earle's modified Eagle's medium supplemented with 10% fetal bovine serum (inactivated at about 56° C.), and supplemented with about 10 g/l of nonessential amino acids, about 1,000 U/ml of penicillin, and about 100 μg/ml of streptomycin.

The splenocytes of such mice are extracted and fused with a suitable myeloma cell line. Any suitable myeloma cell line may be employed in accordance with the present invention; however, it is preferable to employ the parent myeloma cell line (SP2O), available from the ATCC. After fusion, the resulting hybridoma cells are selectively maintained in HAT medium, and then cloned by limiting dilution as described by Wands et al. (Gastroenterology 80:225-232 (1981)), which is herein incorporated by reference. The hybridoma cells obtained through such a selection are then assayed to identify clones which secrete antibodies capable of binding the muscarinic M3 receptor of the invention.

Alternatively, additional antibodies capable of binding muscarinic M3 receptor polypeptide(s) of the present invention can be produced in a two-step procedure using anti-idiotypic antibodies. Such a method makes use of the fact that antibodies are themselves antigens, and therefore, it is possible to obtain an antibody which binds to a second antibody. In accordance with this method, protein specific antibodies are used to immunize an animal, such as a mouse. The splenocytes of such an animal are then used to produce hybridoma cells, and the hybridoma cells are screened to identify clones which produce an antibody whose ability to bind to the muscarinic M3 receptor polypeptide(s) of the present invention protein-specific antibody can be blocked by polypeptide(s) of the invention. Such antibodies comprise anti-idiotypic antibodies to the polypeptide(s) of the invention protein-specific antibody and are used to immunize an animal to induce formation of further muscarinic M3 receptor polypeptide(s) of the present invention protein-specific antibodies.

For in vivo use of antibodies in humans, an antibody can be “humanized”. Such antibodies can be produced using genetic constructs derived from hybridoma cells producing the monoclonal antibodies described above. Methods for producing chimeric and humanized antibodies are known in the art and are discussed herein. (See, for review, Morrison, Science 229:1202 (1985); Oi et al., BioTechniques 4:214 (1986); Cabilly et al, U.S. Pat. No. 4,816,567; Taniguchi et al, EP 171496; Morrison et al., EP 173494; Neuberger et ah, WO 8601533; Robinson et al., WO 8702671; Boulianne et al., Nature 312:643 (1984); Neuberger et al., Nature 314:268 (1985); which are all herein incorporated by reference)

To produce single-change Fv fragments specific for the M3 receptor, naturally occurring V7-genes isolated from human PBLs are constructed into a library of antibody fragments which contain reactivities against muscarinic M3 receptor polypeptide(s) of the present invention to which the donor may or may not have been exposed (see e.g., U.S. Pat. No. 5,885,793 herein incorporated by reference). A library of scFvs is constructed from the RNA of human PBLs as described in PCT publication WO 92/01047. To rescue phage displaying antibody fragments, E. coli harboring the phagemid are used to inoculate 50 ml of 2× TY containing 1% glucose and 100 μg/ml of ampicillin (2× TY-AMP-GLU) and grown to an O.D. of 0.8 with shaking. Five ml of this culture is used to innoculate 50 ml of 2X TY-AMP-GLU, 2× 108 TU of delta gene 3helper (M13 delta gene III, see PCT publication WO 92/01047) are added and the culture incubated at 37° C. for 45 minutes without shaking and then at 37° C. for 45 minutes with shaking. The culture is centrifuged at 4000 r.p.m. for 10 min. and the pellet resuspended in 2 liters of 2× TY containing 100 Ag/ml ampicillin and 50 ug/ml kanamycin and grown overnight. Phages are prepared as described in PCT publication WO 92/01047.

Immunotubes (Nunc) are coated overnight in PBS with 4 ml of either 100 μg/ml or 10μg/ml of a polypeptide of the present invention. Tubes are blocked with 2% Marvel-PBS for 2 hours at 37° C. and then washed 3 times in PBS. Approximately 1000 TU of phage is applied to the tube and incubated for 30 minutes at room temperature tumbling on an over and under turntable and then left to stand for another 1.5 hours. Tubes are washed 10 times with PBS 0.1% Tween-20 and 10 times with PBS. Phages are eluted by adding 1 ml of 100 mM triethylamine and rotating 15 minutes on an under and over turntable after which the solution is immediately neutralized with 0.5 ml of 1.0M Tris-HCl, pH 7.4. Phages are then used to infect 10 ml of mid-log E. coli TG1 by incubating eluted phage with bacteria for 30 minutes at 37° C. The E. coli are then plated on TYE plates containing 1% glucose and 100 g/ml ampicillin. The resulting bacterial library is then rescued with M3 gene helper phage as described above to prepare phage for a subsequent round of selection. This process is then repeated for a total of 4 rounds of affinity purification with tube-washing increased to 20 times with PBS, 0.1% Tween-20 and 20 times with PBS for rounds 3 and 4.

Eluted phage from the 3rd and 4th rounds of selection are used to infect E. coli HB 2151 and soluble scFv is produced (Marks, et al, 1991) from single colonies for assay. ELISAs are performed with microtitre plates coated with either 10 pg/ml of the polypeptide of the present invention in 50 mM bicarbonate pH 9.6. Clones positive in ELISA are further characterized by PCR fingerprinting (see, e.g., PCT publication WO 92/01047) and then by sequencing. These ELISA positive clones may also be further characterized by techniques known in the art, such as, for example, epitope mapping, binding affinity, receptor signal transduction, ability to block or competitively inhibit antibody/antigen binding, and competitive agonistic or antagonistic activity.

The nucleic acid sequences that may be used to attenuate M3 receptor activity or expression include, but are not limited to, RNA, DNA, double-stranded RNA or combinations thereof. In one particular embodiment, the nucleic acids include, but are not limited to, antisense oligonucleotides, small interfering RNA or combinations thereof.

In one embodiment, antisense sequences are generated internally by the organism. In another embodiment, the antisense sequences are separately administered (see O'Connor, Neurochem., 56:560 (1991) and “Oligodeoxynucleotides as Antisense Inhibitors of Gene Expression”, CRC Press, Boca Raton, Fla. (1988), which are both herein incorporated by reference. Antisense technology can be used to control gene expression through antisense DNA or RNA, or through triple-helix formation. Antisense techniques are also discussed, for example, in Okano, Neurochem., 56:560 (1991) which is herein incorporated by reference. Triple helix formation is discussed in, for instance, Lee et al., Nucleic Acids Research, 6:3073 (1979), Cooney et al, Science, 241:456 (1988), and Dervan et al, Science, 251:1300 (1991), all of which are incorporated by reference.

Antagonists, according to the present invention, may also include catalytic RNA, or a ribozyme (See, e.g., PCT International Publication WO 90/11364, published Oct. 4, 1990; Sarver et al, Science, 247:1222-1225 (1990); which are both herein incorporated by reference). While ribozymes that cleave mRNA at site specific recognition sequences can be used to destroy mRNAs corresponding to the polynucleotides of the invention, the use of hammerhead ribozymes is preferred. Hammerhead ribozymes cleave mRNAs at locations dictated by flanking regions that form complementary base pairs with the target mRNA. The construction and production of hammerhead ribozymes is well known in the art and is described more fully in Haseloff and Gerlach, Nature, 334:585-591 (1988), which is herein incorporated by reference. The ribozyme is engineered so that the cleavage recognition site is located near the 5′ end of the mRNA corresponding to the polynucleotides of the invention; i.e., to increase efficiency and minimize the intracellular accumulation of non-functional mRNA transcripts.

As in the antisense approach, the ribozymes of the invention can be composed of modified oligonucleotides (e.g. for improved stability, targeting, etc.) and should be delivered to cells which express the polynucleotides of the muscarinic M3 receptors of the present invention

in vivo,

The nucleic acid sequences used in regulating the expression or activity of the M3 receptors may be operatively linked to a promoter or any other genetic elements necessary to affect the expression of the muscarinic M3 receptor in the target tissue. Such therapy and delivery techniques for nucleic acid sequences are known in the art, see, for example, WO90/11092, WO98/11779; U.S. Pat. Nos. 5,693,622, 5,705,151, 5,580,859; Tabata et al., Cardiovasc. Res. 35(3):470-479 (1997); Chao et al., Pharmacol. Res. 35(6):517-522 (1997); Wolff, Neuromuscul. Disord. 7(5):314-318 (1997); Schwartz et al., Gene Ther. 3(5):405411 (1996); Tsurumi et al., Circulation 94(12):3281-3290 (1996); all of which are herein incorporated by reference.

DNA constructs encoding the ribozyme may be introduced into the cell using well-known techniques. A preferred method of delivery involves using a DNA construct “encoding” the ribozyme under the control of a strong constitutive promoter, such as, for example, pol III or pol II promoter, so that trans feet ed cells will produce sufficient quantities of the ribozyme to destroy endogenous messages and inhibit translation. Since ribozymes, unlike antisense molecules, are catalytic, a lower intracellular concentration may be required for efficiency.

Other compounds that may be used in conjunction with the present invention include, but are not limited to bile acids. Examples of bile acids include, but are not limited to, deoxycholic acid, lithocholic acid and combinations thereof.

Bile acid-induced FXR activation (in association with retinoid X receptor, RXR) alters bile acid synthesis and absorption (primarily ileal component of enterohepatic circulation). Some effects depend on FXR-mediated induction of SHP-1; a small heterodimeric src homology-2 domain-containing Tyr phosphatase. Repression of cholesterol 7α hydroxylation (↓CYP7A1 activity) is mediated by SHP-dependent and -independent mechanisms. With increased bile acid levels, FXR in intestinal epithelium, a bile acid sensor, causes feedback repression of bile acid synthesis (↓CYP7A1 expression). Bile acid-induced activation of FXR also increases expression of enterocyte bile acid binding proteins (e.g. ileal bile acid-binding protein (IBABP)). In vitro studies indicate that potency of deoxycholic acid interaction with FXR is similar to that for interaction with M₃R. FXR^{-/-} mice have negligible expression of ileal bile acid-binding protein (IBABP) and increased Cyp7al activity. IBABP, expressed primarily in ileal enterocytes plays several roles, including facilitation of bile acid uptake and trafficking. In Caco-2 cancer cells, deoxycholic acid robustly increases FXR-mediated induction of IBABP expression. These findings and co-localization of FXR and IBABP in ileal enterocytes provide strong evidence that FXR plays a physiological role in regulating ileal bile acid transport. Other FXR-induced transcription factors that play a role in bile acid homeostasis include HNF1α HNF4 α and organic anion transporting polypeptides (e.g. OATP4 (Slc21a6) which is expressed in mouse liver but not intestine). HNF1 α and HNF4 α bind the bile acid responsive element (BARE) of the CYP7A1 gene, thereby repressing CYP7A1 expression. De Gottardi et al. reported 5- and 10-fold reduction in FXR rnRNA levels in human colon adenomas and carcinomas, respectively. Expression of IBABP was increased (approximately 5-fold) in adenomas compared to control mucosa, and IBABP expression was increased almost 40-fold in some adenocarcinomas.

The agents used in the methods of the present invention may be administered to a subject in the curative, palliative, or prophylactic treatment of cancer. In carrying out the methods of the present invention, the various agents maybe administered to subjects incorporated alone or in a conventional systemic dosage form, such as a tablet, capsule, elixir or injectable. The dose administered must be carefully adjusted according to age, weight and condition of the patient, as well as the route of administration, dosage form and regimen and the desired result. For human use, the agents may be administered alone, but will generally be administered in admixture with a pharmaceutical carrier selected with regard to the intended route of administration and standard pharmaceutical practice.

Alternatively, the agents may be administered by any method that delivers injectable materials to the cells of an animal, such as, injection into the interstitial space of tissues (heart, muscle, skin, lung, liver, intestine and the like). The agents may be delivered in the presence of a pharmaceutically acceptable liquid or aqueous earner, or in the absence of a pharmaceutically acceptable liquid or aqueous carrier. For example, the nucleic acid sequences may be naked nucleic acid sequences in which the term “naked” DNA or RNA, refers to sequences that are free from any delivery vehicle that acts to assist, promote, or facilitate entry into the cell, including viral sequences, viral particles, liposome formulations, lipofectin or precipitating agents and the like. The nucleic acid sequence of the present invention may also be delivered in liposome formulations (such as those taught in Feigner P. L. et al. (1995) Ann. NY Acad. Sci. 772:126-139 and Abdallah B. et al. (1995) Biol. Cell 85(1):1-7, which are both herein incorporated by reference) which can be prepared by methods well known to those skilled in the art.

In accordance with the methods of the present invention, the agent may be administered to any subject in need of treatment for tumor proliferation or tumor metastasis. The subject may be any animal that expresses the M3 receptor, such as a mammal, including human and non-human primates. In one embodiment, the methods of the present invention may be used to administer an agent to a subject who is genetically pre-disposed to tumor development. The tumor development may occur in any organ including, but not limited to, the brain, lungs, stomach, or colon.

The present invention may effective for the treatment of cancers including, but not limited to, acute lymphoblastic leukemia, acute myeloid leukemia, adrenocortical carcinoma, AIDS-related cancers, AIDS-related lymphoma, astrocytoma, basal cell carcinoma, brain tumor, breast cancer, Burkitt's lymphoma, bronchial adenomas, chronic myelogenous leukemia, colon cancer, colorectal cancer, endometrial cancer, esophageal cancer, extrahepatic bile duct cancer, glioma, germ cell tumor, hairy cell leukemia, Hodgkin's lymphoma, islet cell carcinoma, kidney cancer, laryngeal cancer, lung cancer, melanoma, mesothelioma, myeloma, neuroblastoma, non-small cell lung cancer, oral cancer, ovarian cancer, pancreatic cancer, parathyroid cancer, pituitary cancer, prostate cancer, rectal cancer, retinoblastoma, renal cell cancer, salivary gland cancer, small cell cancer, soft tissue sarcoma, stomach cancer, squamous cell carcinoma, T-cell lymphoma, thyroid cancer, uterine cancer, vaginal cancer, or Wilm's tumor.

The agents or procedures are designed to interfere with the M3 receptir induced transactivation of at least one epidermal growth factor receptor (EGFR) on the surface of the cells. As used herein, EGFR is a generic term that is used to mean any member of the family of EGF receptors. In general, EGF receptors, once activated, trigger intracellular events through tyrosine kinase activity. Cascades that EGF receptors are known to trigger include, but are not limited to, the MAPK, Akt and JNK pathways. Accordingly, “transactivation” of an EGF receptor is used to mean the triggering of EGF receptor-induced cascades, where the initial event causing the cascade is the binding of a ligand to a muscarinic receptor.

In a particular embodiment, the methods of the present invention may be used to treat a subject who has a genetic predisposition to cancer, such as a subject with Familial Adenomatous Polyposis or a phenotypically similar disorder.

While the present invention has been described in terms of its specific embodiments, certain modifications and equivalents will be apparent to those skilled in the art and are intended to be included within the scope of the present invention.

The following examples are presented for illustrative purposes only and are not to be construed as limitations for the disclosed invention.

### EXAMPLES

### Example 1

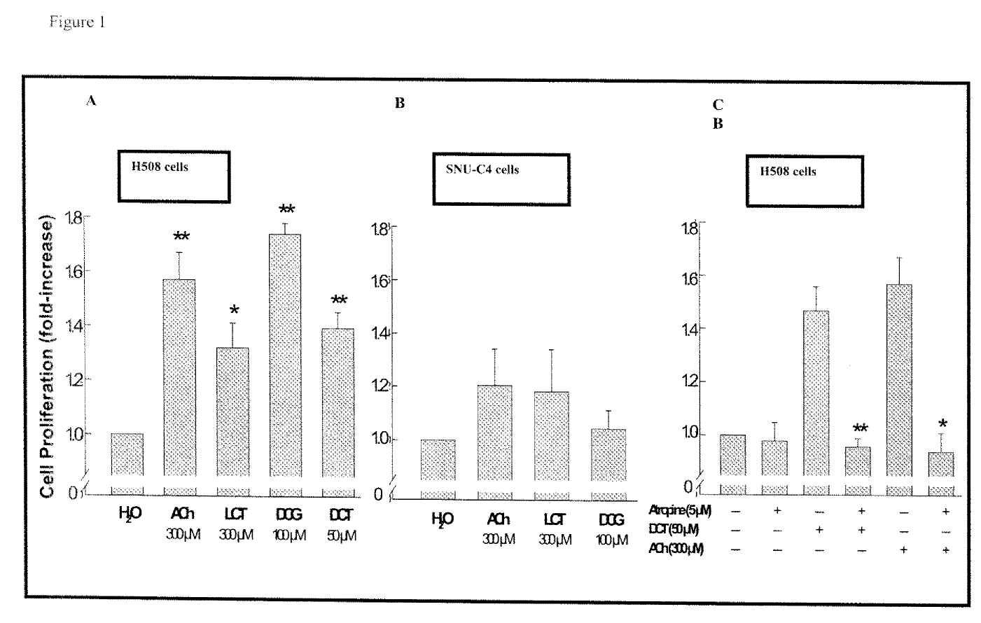

Cells that are known to express both EGFR and M3R were treated with several M3R antagonists and bile acid conjugates to determine if M3R ligand binding affected EGFR activation. H508 cells (A,C) and SNU-C4 cells (B) were treated with ACh, LCT, DCG, or DCT (5 days, 37° C.) alone (A,B) or with atropine (C). Cell proliferation was determined using the sulforhodamine B (SRB) colorimetric assay. As shown in FIG. 1, ACh, lithocholic and deoxycholic acid conjugates stimulate proliferation of H508 cells that co-express M₃R and EGFR, but do not significantly alter proliferation of SNU-C4 cells that express EGFR but not M₃R. As observed, actions of DCT are inhibited by atropine. Mean±SEM of 3 exp. (A): *,** significantly greater than water (p<0.05 and 0.001, respectively, unpaired Student's Mest). (C): *,** significantly less than DCT or ACh alone (p<0.05 and 0.001, respectively).

### Example 2

WT and M₃R^{-/-} male mice (129S6/SvEvTac×CFl; 50%/50%) were treated for 6 wks in which 48 animals received weekly intraperitoneal (ip) PBS (control) or AOM (10 mg/kg). The AOM mouse model is a well-established, commonly used model that mimics human colon carcinogenesis. AOM, an intermediate in the metabolism of 1,2-dimethylhydrazine to the alkylating ion methyldiazonium, is a well-known colorectal-specific pro carcinogen. AOM-induced rodent tumors mimic human colon cancer in that most tumors arise in the colon, form grossly visible exophytic polypoid or plaque-like growths, and have a microscopic appearance similar to that of human lesions. Molecular changes in AOM-induced tumors, including β-catenin and p53 mutations, which also mimic those in human colon cancer. To determine tumor incidence, mice were observed for a full 20 wks. Short-term study end-points are identified (e.g. aberrant crypt foci (ACF), β-catenin-accumulating crypts (BCAC) and BrdUrd staining) and potential toxicity was evaluated, a smaller group of mice is euthanized early, upon completion of AOM treatment.

During the study, WT and M₃R^{-/-} mice were given weekly (for 6 weeks) intraperitoneal (ip) PBS (control; n=10 WT-PBS mice) or AOM (10 mg/kg; n=22 WT-AOM and 16 WT and M₃R^{-/-}-AOM mice). Mice were observed for 14 additional weeks after AOM or PBS treatment for tumor incidence. Animals were housed under identical conditions and fed standard mouse chow. Mice were weighed weekly, observed for evidence of tumor formation (e.g., anal bleeding) and euthanized at 20 weeks. Colon length was measured, segments opened longitudinally, and tumors identified and photographed using a Nikon SMZ1500 dissecting microscope. Tumor number and diameter were measured using Image-Pro software and tumor volume was calculated: vol=½ width×length².

At baseline M₃R^{-/-} mice weighed less than WT mice (20.7±0.5 vs. 28.3±0.5 g; mean±SE; p<0.001) and although both groups gained weight over the 20-wk study period, AOM-treated WT mice persistently weighed ˜16% more than M₃R^{-/-} mice (33.4±1.0 vs. 27.9±0.5 g; at wk 20, p<0.001,). Colon length in M₃R^{-/-} mice was slightly, but significantly, less than that in WT mice (11.6±0.2 vs. 10.7±0.2 cm; WT-AOM compared to M₃R^{-/-}-AOM; p=0.002). M₃R deficiency resulted in a striking 79% reduction in the number of colon tumors/animal. Likewise, tumor size in M₃R^{-/-} mice was reduced. These results indicate that reduction in M3R activity reduces the proliferation of colonic tumor cells. These data also indicate that reduction in M₃R activity also reduces tumor initiation in the colon.

Tissue from WT mice that were treated with weekly AOM for 6 wks and sacrificed at 20 wks was examined histologically. Robust expression of anti-apoptotic proteins (Bcl-X₁ and Bcl-2) was observed in colon gland epithelial cells, although there was less evident expression of pro-apoptotic markers (Bax and Bak),

Expression of cyclin-D1 in colon epithelium was determined from AOM-treated WT mice sacrificed at 20 wks (cyclin-D1 antibody from Santa Cruz). Cyclin-D1 immunostaining was seen clearly in glandular colon epithelial cells, whereas control tissue slides show no immunostaining. All micrographs have embedded size bars. Quantitative analysis of immunohistochemistry data is performed by investigators masked to treatment group who count stained colon epithelial cells in 10 high power fields.

Two hours before euthanasia, mice received intraperitoneal injection of BrdU (Sigma-Aldrich) (50 mg/kg) to label S-phase cells, a marker of proliferation. BrdU labeling was determined after immunostaining with anti-BrdU antibody (BD Bioscience) by counting BrdU-positive nuclei in 1000 cells (data expressed as percent of total cells that were BrdU-positive). As a marker of apoptosis, we used immunostaining with anti-activated caspase-3 antibody (Cell Signaling Technology). Only complete crypts were evaluated and investigators were masked to treatment group. For analysis of both BrdU and activated caspase-3 staining, only tissue from the distal half of the colon was examined.

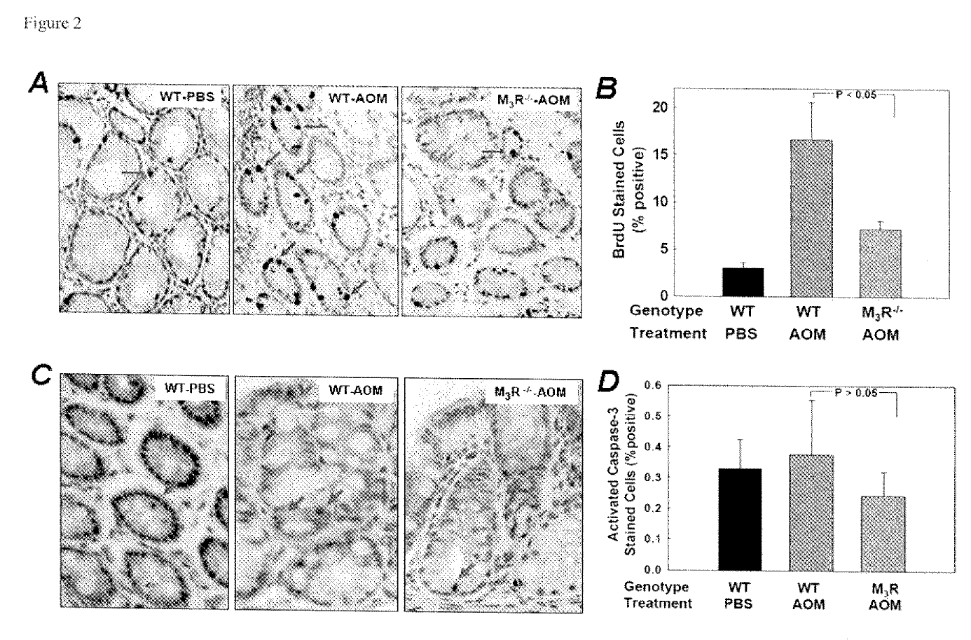

FIG. 2 shows data for BrdU incorporation in colon mucosa from PBS-treated WT, and AOM-treated WT and M3R−/− mice. AOM treatment induced BrdU incorporation in normal colon crypts (FIG. 2A). A 5-fold increase in BrdU-positive epithelial cells was detected in tissue from AOM-treated WT mice and this was reduced in M3R−/− animals (FIG. 2B). FIG. 2B also shows about 70% reduction in BrdU-positive cells in tissue from M₃R−/− compared to WT mice (7.2±0.9% vs. 16.6±4.0%, P<0.05). Clearly, AOM treatment stimulates proliferation of normal colon epithelial cells and this effect is attenuated in M3R-deficient mice.

As a marker of apoptosis, sections of colon mucosa from PBS-treated WT, and AOM-treated WT and M3R−/− mice were examined for activated caspase-3 immunostaining (representative micrographs shown in FIG. 2C). As depicted in FIG. 2D, the number of apoptotic cells in the three treatment groups was an order of magnitude lower than observed with BrdU staining. There was no significant difference in the number of apoptotic cells between groups (P>0.05). These findings indicate that AOM primarily stimulates epithelial cell proliferation and that genetic ablation of M3R reduces cell proliferation with no appreciable effect on apoptosis.

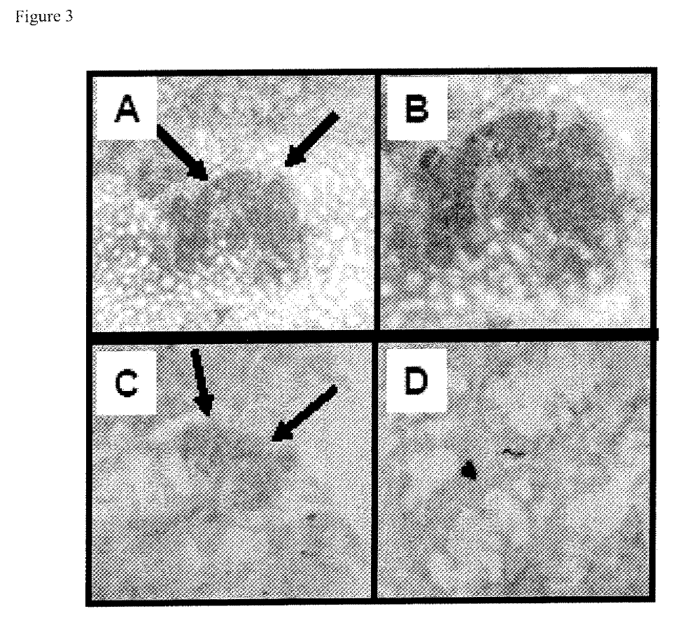

Paraffin-embedded formalin-fixed colon sections were heated at 60° C. overnight, deparaffinnized and rehydrated in xylene, graded alcohol and water. Endogenous peroxide activity was blocked (3% H₂O₂ for 10 min at room temp). After incubating in 0.1% pepsin for 20 min, 1% BSA for 1 hr and 2% normal serum for 1 hr, sections were incubated overnight at 4° C. with (3-catenin antibody (Upstate). Biotinylated secondary antibodies were added for 30 min at room temp. D: BCAC staining control without addition of primary antibody. FIGS. 3A and B shows an example of methylene blue-stained ACF in colon resected from AOM-treated WT mouse. This ACF, shown at low-(A) and high-power magnification (B), has >20 aberrant crypts. FIGS. 3C and 3D shows BCAC stained by immunohistochemistry. ACF and BCAC staining allow identification of early markers of epithelial neoplasia. Formalin-fixed colon from AOM-treated WT mouse was used to detect ACF (0.25% methylene blue) (A+B) and BCAC (C+D). A+B: ACF (arrows) at X40 and X100 original magnification, respectively. C: BCAC (arrows), X100.

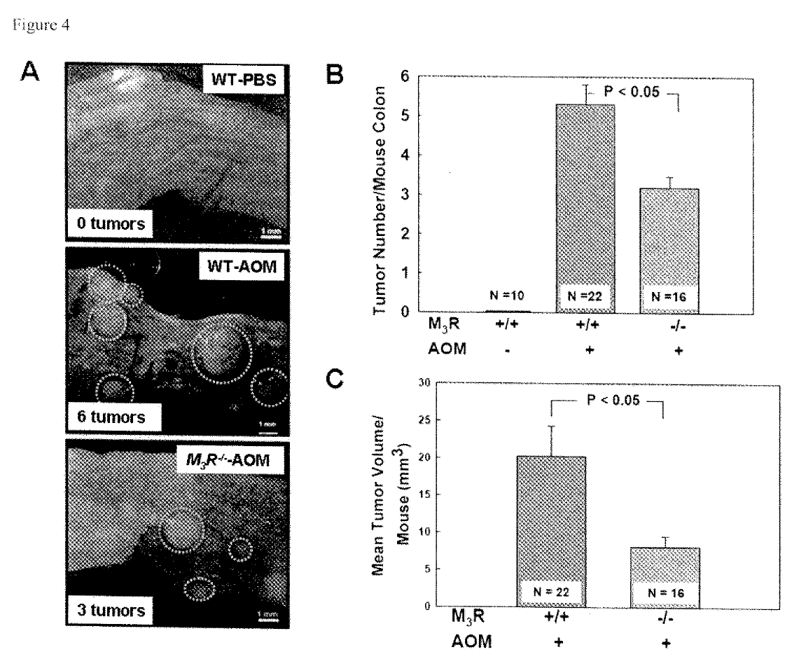

Representative colon sections from AOM-treated animals shown in FIG. 4A indicate reduced tumor number in AOM-treated M3R−/− compared to WT mice. At 20 weeks, no colon tumors were observed in WT mice that had not been treated with AOM. Likewise, colon tumors were not observed in M3R−/− mice that were not treated with AOM. As illustrated in FIG. 4B, WT mice treated with AOM had 5.3±0.5 tumors/colon whereas M3R−/− mice had 3.2±0.3 tumors/colon, which was a 40% reduction (P<0.05). In both WT and M3R-deficient mice, all tumors were in the distal half of the colon. Tumor volume was reduced by 60% in M3R−/− compared to WT mice (8.1±1.5 vs. 20.3±4.1 mm³; P<0.05) (FIG. 4C). These findings provide strong evidence that M3R gene ablation decreases both colon tumor number and size.

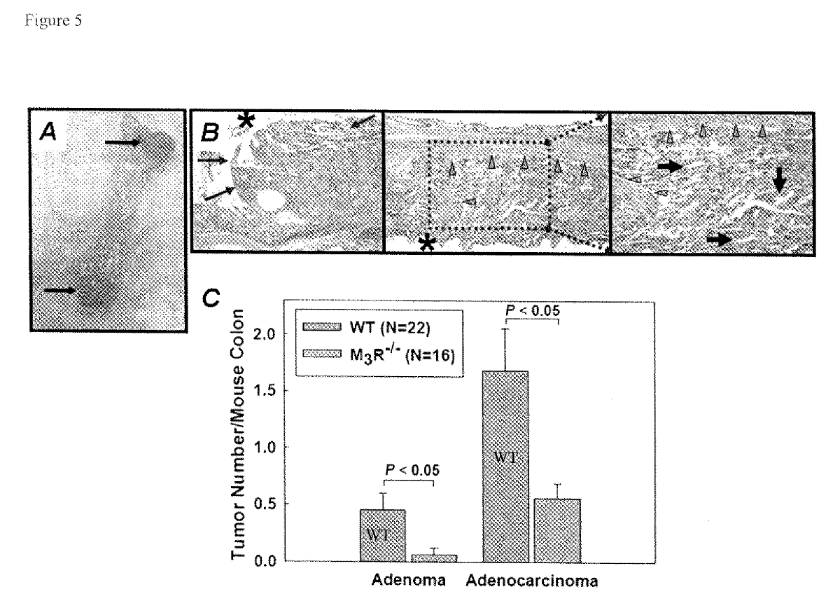

AOM-induced colon tumors mimicked the gross (FIG. 5A) and microscopic (FIG. 5B) appearance of human adenomas and adenocarcinomas. Tumors observed in AOM-treated mice included adenomas (FIG. 5B, left panel),well differentiated adenocarcinomas and poorly differentiated adenocarcinomas (FIG. 5B, middle and right panels). To determine the relative number of adenomas and adenocarcinomas, hematoxylin and eosin-stained colon sections were reviewed by a pathologist masked to treatment group (22 WT and 16 M3R−/− mice treated with AOM). This analysis revealed that, compared to WT, fewer M3R−/− mice had adenomas (6% vs. 36%, P=0.05) and M3R−/− mice had fewer adenocarcinomas/mouse (0.6±0.1 vs. 1.7±0.4, P<0.05) (FIG. 5C). Eleven of 22 WT but no M3R−/− mice had multiple adenocarcinomas (P<0.001). Collectively, these findings indicate that in this murine model of colon cancer, M3R− activity attenuates the overall number of colon tumors and, more specifically, the number of adenomas and adenocarcinomas.

### Example 3

Deoxycholyltaurine(DCT) conjugates stimulate colon cancer cell muscarinic signaling and proliferation

MAPK activation was detected with 10 μM and maximal with 300 μM DCT. The proliferation of H508 cells, which are cells that express muscarinic M3 receptors, was stimulated after 5 days and was detected over the same range of concentrations that activates MAPK. DCT-induced MAPK activation is rapid and reversible. MAPK activation is detected within 1 min, maximal by 10-20 min and returns to basal by 70 min. Collectively these findings emphasize the importance of co-expression of M₃R and EGFR for both ACh- and bile acid-induced colon cancer cell proliferation, and define relevant DCT concentrations.

### Example 4

Muscarinic receptor subtypes were expressed in murine gastric chief cells using M₁R^{-/-}, M₃R^{-/-} and M_{1/3}R^{-/-} mice. To determine the role of M₁R and M₃R in mediating carbachol-induced pepsinogen secretion, M₁R and M₃R-null mice were used. The mice were generated using gene targeting. There was an approx. 25% decrease in maximal carbachol-induced secretion from M₁R^{-/-} mouse gastric glands. In contrast, there was no difference in carbachol potency or efficacy when comparing M₃R^{-/-} to WT mouse gastric glands. To explore the possibility that chief cells express both M₁R and M₃R and that one subtype compensates for absence of the other, the actions of carbachol on pepsinogen secretion were examined from gastric glands prepared from dual M_{1/3}R^{-/-} mice. Strikingly, carbachol-induced secretion was eliminated in glands from M_{1/3}R^{-/-} mice whereas secretion with cholecystokinin (CCK), an agonist that interacts with a different class of receptors (CCK₁R), was not altered. Moreover, in situ hybridization using digoxigenin-labeled antisense RNA probes confirmed co-expression of M₁R and M₃R mRNA in WT gastric glands.

Antisense RNA probes for in situ hybridization to determine expression of muscarinic receptor subtypes. Digoxigenin-labeled antisense RNA probes were prepared from riboprobe plasmids containing M1R and M3R inserts. M1R riboprobe was synthesized from a 0.28-kb KpnI-SacI genomic fragment cloned into a pBluescript vector corresponds to the M1R sequence lacking in the genome of M1R−/− mice. M3R riboprobe was synthesized from a 1.6-kb XbaI-Sse8337I genomic fragment corresponds to M3R sequence absent in genome of M3R−/− mice. M1R and M3R riboprobes were digested with KpnI and NotI, respectively. After purification of linearized plasmids, in vitro transcriptions for M1R and M3R RNA probes, 280 bp and 1.6 kb in length, respectively, were performed using the Digoxigenin RNA labeling kit (Roche Applied Sciences) with T7 and T3 RNA polymerases, respectively. M3R digoxigenin-labeled RNA was shortened by alkaline hydrolysis to ˜300 bp. Yield of transcripts was estimated using dot blots with control digoxigenin-labeled RNA (Roche Applied Science).

### Example 5

The role of M₃R, EGFR, and bile acid nuclear receptors in mediating bile acid-induced colon neoplasia.

To determine the role of M₃R expression in mediating colon carcinogenesis, alone and in the presence of added bile acids, two murine colon cancer models can be used. Mice treated with the cancer initiator azoxymethane (AOM) and mice with a mutated Apc gene were used. Deoxycholyltaurine (DCT) is the test bile acid for these studies because in vitro studies indicate that DCT stimulates human colon cancer cell proliferation over ranges of concentrations that are achieved in the proximal human colon and is not likely to stimulate inflammation or apoptosis. In general, DCT treatment increases colon tumor number in carcinogen-treated mice.

The rationale for including both murine colon cancer models in this study is to observe important differences between these cancer models. Although the Apc^{Min/+} genotype mimics more closely the human condition (e.g. FAP), the AOM-treated mouse phenotype is more similar to human colon cancer. Hence, these two different models may be relevant in showing the importance of M₃R expression for colon carcinogenesis. Additionally, Apc^{Min/+} mice provide an opportunity to study the relationship between M₃R and activation and expression of genes in the Wnt/β-catenin signaling pathway. Binding of Wnt to its cell surface receptor (Frizzled) leads to activation of β-catenin (nuclear translocation and binding to Tcf/LEF, resulting in transcription of important target genes, including cyclin D1). Apc, is a scaffold protein that promotes GSK-3α/β-mediated phosphorylation of β-catenin; phosphorylated β-catenin is targeted for proteosomal degradation, thereby preventing nuclear translocation and pro-proliferative gene transcription. Mutated Apc in Apc^{Min/+} mice results in reduced β-catenin phosphorylation, thereby promoting nuclear translocation and pro-proliferative actions. Bile acid treatment of colon cancer cells has pro-proliferative effects on Wnt/β-catenin signaling; deoxycholic acid increases nuclear translocation of β-catenin and reveal that DCT stimulates an inactivating phosphorylation of GSK-3α/β. These findings provide strong evidence that bile acid-activated M₃R signaling can promote pro-proliferative actions of β-catenin. The Apc^{Min/+} mouse provides an excellent model to elucidate the role of M₃R activation and expression in Wnt/β-catenin signaling.

Preliminary studies in bile acid-treated mice did not show overt evidence of mucosal inflammation. Bile acid-induced inflammation could confound interpretation of the role of M₃R in mediating bile acid actions on tumor formation. Several approaches are proposed to assess tissue for bile acid-induced inflammation. Mouse weight, appearance, and the presence of rectal bleeding (overt and occult measured using Hemoccult cards, Beckman Coulter Inc.—graded as 0=no blood, 1=trace blood, 2=positive, and 3=gross blood) will be assessed daily (Mon-Fri). Resected colon length are measured and microscopic sections (from both proximal and distal ½ of colon) examined for evidence of colitis (H+E stained sections will be reviewed and scored for acute inflammatory cells (0-3), chronic inflammatory cells (0-3) and crypt damage (0-4, where 0=intact crypt, 1=loss of basal third o crypt, 2=loss of basal two thirds, 3=loss entire crypt, 4=loss of crypt and surface epithelium), This scoring system was validated in murine colitis models. Quantitative real-time PCR will be used to examine mucosal sections from treated and control WT and M₃R^{-/-} mice for altered expression of molecular markers of inflammation (TNFα, IL-1β, ICAM, IL-6, and MMP-2- normalized to level of cyclophilin mRNA).

To examine the role of M₃R activation in mediating ‘early’ steps in bile acid-induced colonic neoplasia WT and M₃R^{-/-} mice are treated with AOM followed by intrarectal treatment with DCT or vehicle (PBS). Aberrant crypt foci (ACF) and β-catenin-accumulating crypts (BCAC) are epithelial pre-cancerous changes that serve as markers for early stages of colon carcinogenesis. Whether the carcinogenic actions of DCT is M₃R-dependent is measured by development of ACF and BCAC, and histological indices of cell proliferation [5-bromo-2′-deoxyuridine (BrdUrd) labeling] and apoptosis [terminal deoxynucleotidyl transferase-mediated nick end labeling assay (TUNEL)] caused by DCT is evaluated.

Colon tumor number is reduced in M₃R-deficient AOM-treated mice. Comparison of DCT-induced tumor formation in AOM-treated WT and M₃R^{-/-} mice should reveal the role of M₃R in mediating these effects (i.e., increases the number and size of tumors in AOM-treated WT animals in comparison with M₃R-deficient animals). The expression of genes relevant to M₃R and EGFR signaling, and cell proliferation should be increased in DCT-treated WT compared to PBS-treated WT and PBS- and DCT-treated M₃R^{-/-} animals.

After a 2-wk acclimatization, 1- to 2-month old WT and M₃R^{-/-} mice with free access to a standard commercial diet are treated with AOM (10 mg/kg ip weekly×6 wks) and receive either vehicle (0.1 ml PBS) or 0.1 ml 100 μM DCT ir daily Mon-Fri (total=30 days). AOM and DCT are dissolved in fresh PBS 1 hour before instillation. The dose of AOM will be selected from previous colon cancer studies in rodents. Initial DCT dose, selected from studies in H508 colon cancer cells, are altered based on results of dose-ranging experiments. For short-term end-points mice are euthanized, colons resected, and fixed in 10% formalin. Fixed tissue are stained (0.25% methylene blue for ACF and immunohistochemistry for BCAC) for examination.

For the long-term study, mice are sacrificed when they develop overt or occult anal bleeding or at 20 wk if they have not developed overt evidence of colon cancer. Duration of short (6-wk)- and long (20-wk)-term studies is based on published data with the AOM murine colon cancer model. Two hours before euthanasia, mice receive BrdUrd (50 mg/kg i.p.) to label S-phase cells. After euthanasia, colons are excised and processed, cut longitudinally, laid flat on slides to count grossly visible lesions and for digital photography. The number of study animals was determined by calculations described below. At both 6 and 20 wks, activation of genes related to M₃R and EGFR signaling, bile acid nuclear receptors, and to cell proliferation and apoptosis are determined in resected colon by immunoblots and real-time PCR.

Numbers of mice per group are derived from sample size calculations using numbers required to ensure reproducibility of results, based on a statistical power of 0.8 (β error of 0.20 and α error <0.05). To compare effects of short-term treatment of WT and M₃R^{-/-} mice with AOM and DCT, the numbers of ACF and BCAC in each mouse will be used as our outcome measure. In long-term studies, colon tumors are the primary outcome measure. Specific comparisons include: 1) WT mice treated with AOM are compared to mice treated with AOM and DCT; 2) M₃R^{-/-} mice treated with AOM are compared to WT mice treated with AOM; 3) WT mice treated with AOM and DCT are compared to M₃R^{-/-} mice treated with AOM and DCT; and 4) M₃R^{-/-} mice treated with AOM are compared to those treated with AOM and DCT. Under the assumption of equal variance, in the 6-wk study, using 20 mice per group (×4 groups=80 mice) provides about 80% power to detect a large difference (effect size 0.8) at a (one-sided) significant level of 0.05. For example, if the mean of the number of ACF in. WT mice treated with AOM is 2.9 (SD=0.4) this will provide about 80% power to detect a 12% increase in the mean number of ACF in WT mice treated with AOM and DCT. For the long-term, study, with 30 mice per group (×4 groups=120 mice) the study has about 80% power to detect a medium to larger difference in tumor incidence (effect size 0.7) at a (one-sided) significance level of 0.05. If tumor incidence in WT mice treated with AOM is 50%, have 80% power to detect a 20% increase in tumor incidence in WT mice treated with AOM and DCT (60%). Sample size calculations take into account that 10-15% of mice may die prematurely (sedation, test agent toxicity, or unforeseen causes). Results of DCT dose-ranging studies may modify the number of mice in study groups.

Intraepithelial neoplasia will be determined by analyzing colon epithelium for ACF and BCAC. In rodents treated with carcinogens, ACF and BCAC are the earliest identifiable neoplastic lesions. BCAC may be more reliable indicators of neoplasia than ACF. To be certain that validated markers of neoplasia resected colons are examined for both ACF and BCAC. Initial experiments will determine differences in ACF and BCAC comparing WT to M₃R^{-/-} mice following 6-wk treatment with AOM and DCT. For analysis, the proximal and distal halves of the colon are evaluated separately.

After administering IACUC-approved sedation (ketamine/xylosine), the colon is excised using standard techniques and the lumen flushed with iced PBS to remove feces. The colon is cut longitudinally and placed flat on a microscope slide, mucosa side up, and fixed in neutralized 10% formalin overnight and stored in 70% ethanol at −20° C. Whole-mount fixed murine colonic tissue is stained for 30 sec with 0.25% methylene blue, de-stained with cold PBS, and examined for ACF by transillumination using a dissecting microscope (Nikon SMZ1500) at 20- to 40-× magnification. ACF is defined as “one or more crypts larger than most crypts in the field, have a thickened layer of epithelial cells that stain more intensely with methylene blue, often have a slit-shaped luminal opening, have an increased pericryptal space, and are elevated from the focal plane of the microscope”. After evaluation for ACF, tissues will be destained in PBS and BCAC will be identified by immunohistochemistry for β-catenin using standard techniques. Briefly, paraffin-embedded formalin-fixed sections will be deparaffinized, rehydrated, and antigen retrieval performed by microwaving in citrate buffer. Monoclonal β-catenin antibody (Transduction Laboratories), goat anti-mouse antibody, and staining kits are commercially available. Digital images of all lesions are archived.

Laser Capture Microdissection (LCM) may also be used to maximize tumor cell ‘purity’ from specimens for analyses of molecular activation and expression. LCM optimizes the ability to extract mRNA from neoplastic versus normal tissue. To maximize total yield, 5 adjacent tumor sections are pooled.

Normal epithelium adjacent to tumors will be sampled for comparison. Frozen sections are prepared by embedding specimens in OCT compound within a disposable plastic mold and cutting with a cryotome. Before LCM, thin frozen sections are fixed in 75% ethanol for 30 sec, then immediately stained with HistoGene Stain (Arcturus, Mountain View, Calif.). After staining, sections are air-dried for at least 5 min. Cells from each section are selected by applying multiple laser pulses. For each section, 5 LCM caps are generated in turn and immediately immersed in RNA-protecting buffer (Micro RNA Isolation Kit, Stratagene, La Jolla, Calif.). The remaining tumors are analyzed without LCM (i.e., by gross dissection of tumor tissue at resection). Each lesion or histological cell type is separately microdissected, the microdissected area adhered to Eppendorf tube caps using the laser within the LCM device, and RNA extraction performed in Eppendorf tubes. Preliminary data using LCM-dissected mouse tumors indicate that RNA extraction and quantitative RT-PCR is successful using LCM-derived material.

Total RNA is extracted from LCM specimens using the Micro RNA Isolation Kit

(Stratagene). mRNA is amplified for two rounds using the RiboAmp RNA Amplification Kit (Arcturus). This kit amplifies nanogram amounts of starting RNA utilizing a 5-step process for linear amplification of the mRNA fraction of total cellular RNA: 1) a first-strand synthesis reaction yields cDNA incorporating a T7 promoter; 2) a second-strand synthesis reaction utilizing exogenous (proprietary) primers yields double-stranded cDNA; 3) a specially designed purification column purifies the double-stranded cDNA; 4) following cDNA isolation, in vitro transcription utilizing T7 RNA polymerase yields antisense RNA (aRNA); and 5) aRNA is isolated with the purification column. In preliminary experiments, the degree of amplification achieved from high-quality starting RNA is 200,000-fold: for example, 30 ng total RNA, presumed to contain approximately 300 pg mRNA, after two rounds of amplification yields 60 μg aRNA. This amount is sufficient to permit repetition and verification.

Two hours before euthanasia, the mice received i.p. injection of BrdUrd (50 mg/kg) to label S-phase cells, which is an index of proliferation. BrdUrd labeling was determined after immunostaining with anti-BrdUrd (Sigma kit) by counting BrdUrd-positive nuclei in 1000 cells. Immunoblotting for Ki-67, which is expressed only in the S-phase of the cell cycle, also provided a marker of proliferation. As an index of apoptosis, the terminal deoxynucleotidyl transferase-mediated nick end labeling assay (TUNEL, Intergen) was used. For BrdUrd and TUNEL labeling, only complete crypts extending from muscularis mucosa to colon lumen were counted.

Colon tumor number and size and BrdUrd labeling, are analyzed after DCT treatment and compared in WT and M₃R^{-/-} mice. Histological definitions of adenomas and adenocarcinomas conformed to consensus recommendations (Mouse Models of Human Cancers Consortium). The number of tumors is counted in the colon from each mouse and the tumors are photographed, resected and bisected. Half of the tumor are fixed overnight in neutralized 10% formalin at room temperature and processed for histology by standard techniques. The other half are stored in RNAlater (Ambion), and protein and RNA is extracted. The remaining tissue and tissue from colons, where tumors were not detected grossly, are fixed in 4% paraformaldehyde at 4° C. overnight and paraffin-embedded. 5-μm sections are cut every 200 μm through the complete block, stained with hematoxylin and eosin, and examined under a dissection microscope with an in-scope micrometer. Investigators will be masked to mouse genotype and treatment performed gross and microscopic tumor counts and determined tumor size. Tumor volume is calculated from digitized images using Nikon Image-Pro software. For analysis, tumors are stratified by size (calculated volume <2 mm³, 2-3.9 mm³, 4-5.9 mm³, ≧6 mm³). The 20 wk control mice have been reported to develop grossly visible colon tumors (adenomas and adenocarcinomas) at a low ‘background’ rate (approx. 3-5 tumors/animal). Bile acid-treated animals are reported to have at least twice this number of tumors.

Examination of bile acid-induced changes in expression and activation of genes related to M₃R and EGFR signaling, and associated with colon cancer. In AOM-treated and control mice (with and without bile acid infusion), comparison of tumors and adjacent normal tissue, expression and activation of genes related to post M₃R and EGFR signaling, and of key genes that are associated with colon cancer. Preliminary Studies in H508 colon cancer cells, indicate that proliferative effects of muscarinic agonists, including secondary bile acids, depend on activation of both M₃R and EGFR, and these effects are associated with phosphorylation of downstream signaling molecules, including p44/42 MAPK, p90RSK, Akt, and GSK. Examination of changes in activity and expression of genes/gene products will also include those members of the Wnt/β-catenin signaling pathway. Many genes associated with colon carcinogenesis are either members of the Wnt/β-catenin signaling family or targets of nuclear β-catenin (e.g. PPARδ, cyclin D1, COX-2 and MMP-7). Regarding genes associated with colon cancer, an important role for bile acid-induced activation of MMP-7 and increased expression of both MMP-7 and MMP-1 maybe indicated. β-catenin, E-cadherin, cyclin-D1, PPARδ, p53, nitric oxide synthase (NOS), and cyclooxygenase-2 (COX-2) were selected for examination based on recent studies confirming their importance for the development and progression of colon cancer).

The level of expression of genes related to M₃R, EGFR, and bile acid nuclear receptor signaling, cell proliferation and apoptosis is evaluated. Except for disruption of M₃R expression, WT and M₃R^{-/-} mice have the same genetic background (C57BL/6×129SvEv). For example, in AOM-treated mice, a comparison of tumor and adjacent normal tissue expression of FXR, VDR and downstream targets (Shp-1, IBABP, Oatp4) can be performed. For comparison, and to examine effects on IBABP, expression of these genes is examined in normal ileal mucosa obtained from mice in different treatment groups. Quantitative RT-PCR is performed using methods, controls and internal standards. Primers and probe sets are searched for within the manufacturer's recommended conditions (Tm of primers, 58-60° C.; Tₘ of probe, 10° C. higher than that of primers). Probes are labeled with FAM as a reporter at the 5′-end and TAMRA or BH1 as a quencher at the 3′-end. For example, see primers designed for amplification of selected genes for murine bile acid nuclear receptors and their targets (Table 1). At least 4 isoforms of murine FXR have been reported. Consequently, primers for RT-PCR were designed to recognize consensus sequences (Table 1). A similar approach is used to design primers for other bile acid nuclear receptors and downstream targets.

TABLE 1

Primers designed for RT-PCR amplification of
murine genes for bile acid nuclear receptors
and downstream targets. Common name of gene
is followed by (‘official name’). Sequences
may be obtained from GeneBank

| | |
|---|---|
| Gene | Sequence |

| | | |
|---|---|---|
| FXR | Forward | 5′-GCTCTGCTCACAGCGATCGT-3′ |
| (Nrlh4) | Probe | 5′-FAM-ATCCTCTCTCCAGACAGACAATAC |
| | | ATCAAGGACAG-TAMRA-3′ |
| | Reverse | 5′-CTCCTGCAGCTTCTCCACCG-3′ |
| Shp-I | Forward | 5′-TGAAGGGCACGATCCTCTTCA-3′ |
| (NrOb2) | Probe | 5′-FAM-ATGTGCCAGGCCTCCGTGCC- |
| | | TAMRA-3′ |
| | Reverse | 5′-CAGGTGTGCGATGTGGCAG-3′ |
| IBABP | Forward | 5′-TTGAGAGTGAGAAGAATTACGATGAGT |
| (Fabp6) | | T-3′ |
| | Probe | 5′-FAM-ATGAAGCGCCTGGGTCTTCCAGG- |
| | | TAMRA-3′ |
| | Reverse | 5′-CGTCCCCTTTCAATCACGTC-3′ |
| Oatp4 | Forward | 5′-GGGAGTATTCTGACTGCGTTGC-3′ |
| (Slcol b2) | Probe | 5′-FAM-CATTTCTTCATGGGATATTACAGG |
| | | TATGCAACAGA-TAMRA-3′ |
| | Reverse | 5′-TGACTAAACAGGTCAACGTGGAGTT |
| | | A-3′ |
| VDR | Forward | 5′-CCATCTGCATTGTCTCCCCA-3′ |
| (Nrli1) | Probe | 5′-FAM-AGCCATTCAGGACCGCCTATCCAA |
| | | CAC-TAMRA-3′ |
| | Reverse | 5′-GCAGCGGATGTAGGTCTGC-3′ |
| PXR | Forward | 5′-TGGCCGATGTGTCAACCTACA-3′ |
| (Nrli2) | Probe | 5′-FAM-CGTCATCAACTTCGCCAAAGTCAT |
| | | ATCCTACT-TAMRA-3′ |
| | Reverse | 5′-AAAAGTGGCCCCCTTCAGC-3′ |

Immunoblotting and quantitative real time-PCR is used to determine protein activation, and relative protein and gene expression, respectively, in normal colon and tumors obtained from treated and untreated WT and M₃R^{-/-} mice. When antibodies are available, protein expression and cellular redistribution are examined by immunohistochemistry on deparaffinized tissue blocks. For example, to explore internalization and nuclear translocation of activated M₃R, immunohistochemistry on deparrafinized tissue (M₃R antibody available from Research and Diagnostic Methods) is performed. For studies of protein activation/inactivation, the expression of phosphorylated protein (e.g. pAkt, pGSK), recognizing that phosphorylation may either activate (e.g. Akt) or inactivate (e.g. GSK) a protein is examined. For transcription factors, like NF-κB, nuclear translocation and other measures of activation are measured.

Colon tumors develop because of increased cell proliferation, decreased

apoptosis, or both. Deoxycholic acid induces apoptosis. In previous studies, bile acid actions on colon cancer cells are examined by observing caspase-3 activation and found no evidence of DCT-induced apoptosis. Nonetheless, preliminary studies demonstrate that DCT activates post-EGFR PI3K/Akt signaling, including proteins that may affect apoptosis (Bad, NF-κB). Hence, it is important to analyze tissue for changes in apoptosis.

Complementary approaches will determine whether DCT treatment alters

apoptosis. Cells with DNA strand breaks are identified in resected colons by terminal deoxyuridine nucleotidyl nick-end labeling (TUNEL; Apoptag kit, Intergen). Caspases, especially caspase-3 and -7, play important roles in mediating apoptosis. Caspase-3 and -7 activation are examined in deparaffinzed tissue blocks using immuno-histochemistry with commercially available antisera. Immunoblotting and quantitative RT-PCR are used to measure and compare activation and expression of anti-apoptotic [Bcl-2, Bcl-X (Xₛ and X₁)] and pro-apoptotic (Bad, Bak, Bax) proteins and genes in normal colon epithelium and tumors from WT and M₃R^{-/-} mice. Quantitative analysis of immunohistochemistry data are performed by investigators masked to treatment group. The stained colon epithelial cells are counted in 10 high power fields (results expressed as % total, mean±SEM). Results will be validated using quantitative RT-PCR for the same markers of apoptosis.

Activation of bile acid nuclear receptors are determined by comparing expression of downstream molecules (e.g., IBABP) in tissues obtained form bile acid-treated and untreated animals. Immunostaining can be used to examine cellular localization of bile acid receptors (cytoplasmicmuclear ratio). Previous studies describe shift of some bile acid nuclear receptors from cytoplasm to nucleus following exposure to ligands. Hence, a decrease in the cytoplasmicmuclear ratio for a bile acid nuclear receptor following treatment with bile acids indicates activation of the receptor. Antibodies for immunohistochemistry are commercially available [e.g. antibodies for FXR (C-20, Q-20, H-130); PXR (R-14, H-160, A-20) and VDR (N-20, C-20, H-81) are available from Santa Cruz]. To derive cytoplasmicmuclear staining ratio for bile acid receptor proteins, at least 10 high power fields (≧100 cells) are evaluated by investigators masked to treatment group. Cells are scored as to whether immunostaining is primarily cytoplasmic or nuclear. Cytoplasmmuclear ratio is derived from these values.

Experiments using in situ hybridization for M₃R mRNA in mouse gastric mucosa have revealed intense signal in the cytoplasm of chief cells, whereas in parietal cells the signal was primarily nuclear. With riboprobes, in situ hybridization is used to examine and compare cellular localization of mRNA for bile acid nuclear receptors and target molecules in tumor and normal colon from DCT-treated and untreated WT and M₃R-null mice.

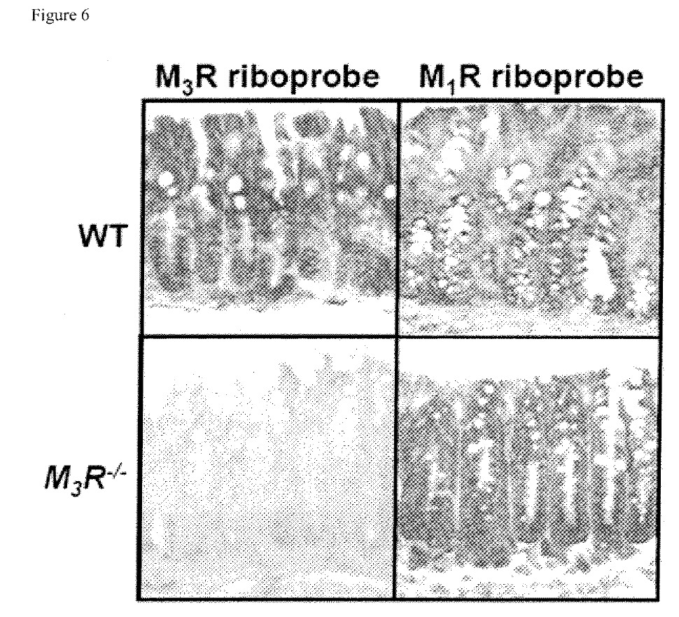

It is possible that, similar to gastric chief cells, mouse colon epithelial ceils co-express M₁R and M₃R, and that both receptors are activated by bile acids. Preliminary in situ hybridization experiments using methods described in reveal that WT mouse colon epithelial cells, in fact, co-express M₃R and M₁R (FIG. 6) shows cytoplasmic M₁R and M₃R mRNA). Hence, bile acids may stimulate tumor growth in M₃R^{-/-} mice by an M₁R-dependent mechanism. Based on studies showing reduced tumor number in M₃R^{-/-} animals, if this is the case, M₁R expression makes only a minor contribution. Nonetheless, in situ hybridization is used to examine muscarinic receptor expression in tumors from DCT-treated WT and M₃R^{-/-} mice. If tumors co-express M₁R and M₃R, and there is not a significant reduction in tumor incidence or size in bile acid-treated M₃R^{-/-} animals, the effects of bile acid treatment on mice that are deficient in both M₁R and M₃R ( M_{1/3}R^{-/-} mice) are examined.

### The Apc Mouse Model

Apc^{Min/+} (multiple intestinal neoplasia) mice are commonly used to study colon cancer. From preliminary studies, colon tumor number and size is reduced in M₃R-deficient AOM-treated mice. Unlike the AOM model, Apc^{Min/+} mice with an inactivating Apc gene mutation similar to that occurring in humans with familial adenomatous polyposis (FAP) do not require treatment with an initiator (e.g. AOM) for tumor formation. In these mice, reduced expression of normal Apc, a molecule in the Wnt/β-catenin signaling pathway, allows β-catenin to accumulate in the nucleus, thereby activating pro-proliferative Tcf/LEF-1 family transcription factors.

After weaning, Apc^{Min/+} mice with variable expression of M₃R were raised with free access to a standard commercial diet. In some studies, Apc^{Min/+} mice were maintained on a high-fat (10%) diet, however consultation with Jackson Labs indicates that their comparison of a high-fat and standard (6% fat) diet revealed no difference in the number of intestinal tumors. The number of mice was determined by sample-size calculations described herein but will be increased if sedatives or frailty results in premature death.

Mice were sacrificed when they develop overt or occult anal bleeding or at an

interval determined from the time-course study if they have not developed evidence of colon cancer. A short-term (6-wk) study was not performed with Apc^{Min/+} mice because there is no concern regarding mucosal inflammation, since animals are not being treated with exogenous bile acids. In addition, ACF and BCAC are rare or absent in these mice.

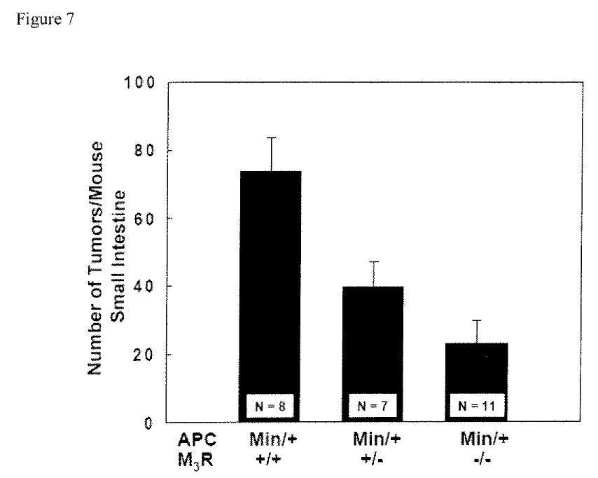

From birth, M₃R^{-/-} mice weigh less than littermate controls indicating that M₃R expression plays a key role in body weight. Apc^{Min/+} mutation did not alter the effect of M₃R on body weight; Apc^{Min/+} M₃R^{-/-} mice (24.5±0.9 g) weighed less at 12 wks than Apc^{Min/+}M₃R^{+/-} mice (30.8±1.1 g) (p<0.01). At 12 wks of age, 7 Apc^{Min/+} M₃R^{+/-} Apc^{Min/+} M₃R^{-/-} mice were euthanized. For control, 8 male Apc^{Min/+} mice with WT M₃R were euthanized. Tumors were identified by direct illumination and transillumination of tissue using a Nikon SMZ1500 dissecting microscope. Male Apc^{Min/+} M₃R^{+/-} and Apc^{Min/+} M₃R^{-/-} mice had a 53% and 79% reduction, respectively, in small intestine tumor number compared to Apc^{Min/+} M₃R^{+/+} mice (p=0.002, Kraskal-Wallis test) (FIG. 7). Analyzing both male and female mice revealed that in Apc^{Min/+} M₃R^{-/-} mice (N=13) small intestine tumor number was reduced by ˜50% compared to Apc^{Min/+} M₃R^{+/-} mice (N=23) (p=0.002, Mann-Whitney U test). Tumor number increased progressively from the proximal to distal third of the small intestine (p<0.001); M₃R ablation reduced tumor number in each segment (p<0.01).

### Example 6

Based on data supporting the important role of M₃R expression in colon tumor

formation, it is possible that using an anti-cholinergic agent may reduce tumor numbers in mice , similar to the reduction observed in mice with lower M₃R expression. For this purpose, scopolamine butylbromide (hyoscine-N-butylbromide) is chosen as the test agent. The rational for this choice was: (1) demonstrated in vitro that bile acid actions are attenuated by treatment with scopolamine, (2) long-term scopolamine administration is commonly used in animal studies when prolonged muscarinic receptor blockade is desired and safe, effective dosing regimens in mice are published, (3) scopolamine blocks all muscarinic receptor subtypes, thereby obviating concerns regarding co-expression in colon epithelial cells of multiple muscarinic receptor subtypes (FIG. 6 shows co-expression of M₁R and M₃R in colon epithelium), (4) as a quaternary ammonium derivative, scopolamine butylbromide does not cross the blood-brain barrier, thereby preventing central nervous system side-effects, (5) administration of scopolamine butylbromide (Buscopan) as a tablet, intravenous solution and dermal patch, is FDA-approved for treatment of muscle spasm of the gastrointestinal tract and has few side-effects in humans and, (6) highly purified scopolamine butylbromide is available for these studies (Sigma-AIdrich, catalog #S7882, ≧99% pure by TLC). The half-life of the terminal elimination phase of scopolamine butylbromide is about 5 hr; protein binding is low, and metabolites are inactive. These features indicate that twice daily subcutaneous dosing in mice is sufficient to maintain prolonged muscarinic receptor blockade,

The two well characterized murine colon cancer models, Apc^{Min/+} and AOM-treated WT mice, are used. To decrease the number of animals required to achieve significant outcomes, only male mice will be used. Both murine colon cancer models are available in our laboratory and demonstrated a significant 5-fold increase in tumors/animal in WT mice treated with AOM and significantly fewer tumors in AOM-treated M₃R^{-/-} mice. Animal survival and intestinal tumor formation and activation and expression of molecular markers of proliferation, apoptosis, and M₃R signaling are compared in Apc^{Min/+} and AOM-treated WT mice treated with scopolamine or PBS. Examination of resected intestine for tumors and altered cell proliferation, apoptosis and M₃R and EGFR signaling. The methods of resecting and examining the intestines for tumors and/or altered cell proliferation are the same as those described above. Similarly, methods of measuring apoptosis, gene expression, immunohistochemistry, RT-PCR and other methods are the same as those described herein.

In selected mice, scopolamine plasma levels are measured using a validated,

sensitive radioreceptor assay. Scopolamine levels may also be measured by other techniques (e.g. HPLC). For these assays, blood from mice just before scopolamine administration (3 mg/kg sc) and at 0.5, 1, 2, 3, 4, 6, 8 and 10 hours after the agent (or PBS control) is administered. The generally accepted desired therapeutic plasma level of scopolamine is >50 pg/ml, maintained over 2-8 hr.

Significance of resulting numerical data are determined using different methods, as appropriate. Significance between means are determined by Student's unpaired t-test with Bonferroni correction for multiple comparisons, or by analysis of variance followed by Dunnett's test, Nonparametric Kruskal-Wallis tests will be used for analysis of variables that are not normally distributed (e.g. comparisons of ACF, BCAC and tumors). ANOVA for a nested design is used to examine treatment effects on BrdUrd labeling. Tukey's procedure is used to adjust for multiple comparisons. Immunoblots and other data that are not numerical are repeated at least 3 times to verify reproducibility. Densitometry is performed on blots for quantification of changes in level of protein or gene expression and compared by un-paired Students t-test. Quantitative analysis of immunohistochemical data is accomplished by investigators masked to treatment group and mouse genotype who will count the number of stained colon epithelial cells in 10 high power fields (expressed as % total; mean±SEM). Values of p<0.05 are considered significant.

The following references are incorporated by reference.

1. Abdallah B, et al, Biol. Cell 1995; 85(1): 1-7.

2. Alleva E, Bignami G. Development of mouse activity, stimulus reactivity, habituation, and response to amphetamine and scopolamine. Physiol Behav 1985;34:519-23,

3. Alpini G, Glaser S S, Ueno Y, Rodgers R, Phinizy J L, Francis H, Baiocchi L, Holcomb L A, Caligiuri A, LeSage G D. Bile acid feeding induces cholangiocyte proliferation and secretion: evidence for bile acid-regulated ductal secretion. Gastroenterology 1999;116:179-86.

4. Baijal P K, Clow E P, Fitzpatrick D W, Bird RP. Tumor-enhancing effects of cholic acid are exerted on the early stages of colon carcinogenesis via induction of aberrant crypt foci with an enhanced growth phenotype. Can J Physiol Pharmacol 1998;76:1095-102.

5. Baijal P K, Fitzpatrick D W, Bird R P. Comparative effects of secondary bile acids, deoxycholic and lithocholic acids, on aberrant crypt foci growth in the postinitiation phases of colon carcinogenesis. Nutr Cancer 1998;31:81-9.

6. Baron J A, Sandler R S, Bresalier R S, Quail H, Riddell R, Lanas A, Bolognese J A, Oxenius B, Horgan K, Loftus S, Morton D G. A randomized trial of rofecoxib for the chemoprevention of colorectal adenomas. Gastroenterology 2006;131:1674-82.

7. Bellacosa A, Kumar C C, Di Cristofano A, Testa J R. Activation of AKT kinases in cancer: implications for therapeutic targeting. Adv Cancer Res 2005;94:29-86.

8. Bertagnolli M M, Eagle C J, Zauber A G, Redston M, Solomon S D, Kim K, Tang J, Rosenstein R B, Wittes J, Corle D, Hess T M, Woloj G M, Boisserie F, Anderson W F, Viner J L, Bagheri D, Burn J, Chung D C, Dewar T, Foley T R, Hoffman N, Macrae F, Pruitt R E, Saltzman J R, Salzberg B, Sylwestrowicz T, Gordon G B, Hawk E T. Celecoxib for the prevention of sporadic colorectal adenomas. N Engl J Med 2006;355:873-84.

9. Bird R P, Salo, D., Lasko, C, Good, C. A novel methodological approach to study the level of specific protein and gene expression in aberrant crypt foci putative preneoplastic colonic lesions by Western blotting and RT-PCR. Cancer Lett 1997;116:15-9.

10. Bird R P. Observation and quantification of aberrant cruypts in the murine colon treated with a colon carcinogemprelirninary findings. Cancer Lett 1987;37:147-51.

11. Boivin G P, Washington K, Yang K, Ward J M, Pretlow T P, Russell R, Besselsen D G, Godfrey V L, Doetschman T, Dove W F, Pitot H C, Halberg R B, Itzkowitz S H, Groden J, Coffey R J. Pathology of mouse models of intestinal cancer: consensus report and recommendations. Gastroenterology 2003;124:762-77.

12. Bonner T, Buckley N, Young A, Brann M. Identification of a family of muscarinic acetylcholine receptor genes. Science 1987;237:527-32.

13. Boulianne, et al., Nature, 1984; 312:643.

14. Brann M R, Ellis J, Jorgensen H, Hill-Eubanks D, Jones SV. Muscarinic acetylcholine receptor subtypes: localization and structure/function. Prog Brain Res 1993;98:121-7.

15. Chao, et al., Pharmacol. Res. 1997; 35(6):517-522.

16. Cheng K, Chen Y, Zimniak P, Raufman J, Xiao Y, Frucht H. Functional interaction of lithocholic acid conjugates with M3 muscarinic receptors on a human colon cancer cell line. Biochim Biophys Acta 2002;1588:48-55.

17. Cheng K, Khurana S, Chen Y, Kennedy R H, Zimniak P, Raufman JP. Lithocholylcholine, a bile acid/acetylcholine hybrid, is a muscarinic receptor antagonist. J Pharmacol Exp Ther 2002;303:29-35.

18. Cheng K, Raufman J P. Bile acid-induced proliferation of a human colon cancer cell line is mediated by transactivation of epidermal growth factor receptors. Biochem Pharmacol 2005;70:1035-47.

19. Cheng K, Xie G, Raufman J P. Matrix metalloproteinase-7-catalyzed release of HB-EGF mediates deoxycholyltaurine-induced proliferation of a human colon cancer cell line Biochemical Pharmacology 2007;In Press (doi: 10.1016/j.bcp.2006.11.028)

20. Cheng K, Zimniak P, Raufman J P. Transactivation of the epidermal growth factor receptor mediates cholinergic agonist-induced proliferation of H508 human colon cancer cells. Cancer Res 2003;63:6744-50.

21. Chiang J Y. Regulation of bile acid synthesis: pathways, nuclear receptors, and mechanisms. J Hepatol 2004;40:539-51.

22. Cintron N M, Chen Y M. A sensitive radioreceptor assay for determining scopolamine concentrations in plasma and urine. J Pharm Sci 1987;76:328-32.

23. Claro F T, Patti C L, Abilio V C, Frussa-Filho R, Silva R H. Bovine brain phosphatidylserine attenuates scopolamine induced amnesia in mice. Prog Neuropsychopharmacol Biol Psychiatry 2006;30:881-6.

24. Cohen G M. Caspases: the executioners of apoptosis. Biochem J 1997;326 (Pt 1):1-16.

25. Cohen J. Statistical Power Analysis for the Behavioral Sciences. New Jersey: Lawrence Erbaum, 1988.

26. Cooney, et al., Science 1988; 243:456.

27. Daub H, Weiss F U, Wallasch C, Ullrich A. Role of transactivation of the EGF receptor in signalling by G-protein-coupled receptors. Nature 1996;379:557-60.

28. De Gottardi A, Touri F, Maurer C A, Perez A, Maurhofer O, Ventre G, Bentzen C L, Niesor E J, Dufour J F. The bile acid nuclear receptor FXR and the bile acid binding protein IBABP are differently expressed in colon cancer. Dig Dis Sci 2004;49:982-9.

29. Dervan, et al., Science 1991; 251:1300.

30. Ding Q, Xia W, Liu J C, Yang J Y, Lee D F, Xia J, Bartholomeusz G, Li Y, Pan Y, Li Z, Bargou R C, Qin J, Lai C C, Tsai F J, Tsai C H, Hung M C. Erk associates with and primes GSK-3beta for its inactivation resulting in upregulation of beta-catenin. Mol Cell 2005;19:159-70.

31. Earnest D L, Holubec H, Wali R K, Jolley C S, Bissonette M, Bhattacharyya A K, Roy H, Khare S, Brasitus T A. Chemoprevention of azoxymethane-induced colonic carcinogenesis by supplemental dietary ursodeoxycholic acid. Cancer Res 1994;54:5071-4.

32. Estape N, Steckler T. Cholinergic blockade impairs performance in operant DNMTP in two inbred strains of mice. Pharmacol Biochem Behav 2002;72:319-34.

33. Feigner P L. et al., Ann. NY Acad. Sci. 1995; 772:126-139.

34. Feidstein A E, Canbay A, Angulo P, Taniai M, Burgart L J, Lindor K D, Gores G J. Hepatocyte apoptosis and fas expression are prominent features of human nonalcoholic steatohepatitis. Gastroenterology 2003;125:437-43.

35. Fisahn A, Yamada M, Duttaroy A, Gan J W, Deng C X, McBain C J, Wess J. Muscarinic induction of hippocampal gamma oscillations requires coupling of the M1 receptor to two mixed cation currents. Neuron 2002;33:615-24.

36. Frucht H, Gazdar, A. F., Park, J- A., Oie, E L, Jensen, R. T. Characterization of functional receptors for gastrointestinal hormones on human colon cancer cells. Cancer Res 1992;52:1114-22.

37. Frucht H, Jensen, R. T., Dexter, D., Yang, W- L., Xiao, Y. Human colon cancer cell proliferation mediated by the M3 muscarinic cholinergic receptor. Clin Cancer Res 1999;5:2532-9.

38. Grober J, Zaghini I, Fujii H, Jones S A, Kliewer S A, Willson T M, Ono T, Besnard P. Identification of a bile acid-responsive element in the human ileal bile acid-binding protein gene. Involvement of the farnesoid X receptor/9-cis-retinoic acid receptor heterodimer. J Biol Chem 1999;274:29749-54.

39. Guda K, Giardina C, Namibiar P R, Cui H, Rosenberg D W. Aberrant transforming growth factor-beta signaling in azoxymethane-induced mouse colon tumor. Mol Carcinog 2001 ;31:204-13.

40. Hammerling, et al., in: Monoclonal Antibodies and T-Cell Hybridomas, Elsevier, N.Y., pp. 563-681, 1981.

41. Haseloff and Gerlach, Nature 1988; 334:585-591.

42. Heimbrook D C, Stirdivant S M, Ahern J D, Baiishin N L, Patrick D R, Edwards G M, Defeo-Jones D, FitzGerald D J, Pastan I, Oliff A. Transforming growth factor alpha-Pseudomonas exotoxin fusion protein prolongs survival of nude mice bearing tumor xenografts. Proc Natl Acad Sci USA 1990;87:4697-701.

43. Hill M J, Drasar B S, Williams R E, Meade T W, Cox A G, Simpson J E, Morson B C. Faecal bile-acids and Clostridia in patients with cancer of the large bowel. Lancet 1975;1:535-9.

44. Hill M J. Bile acids and colorectal cancer: hypothesis. Eur J Cancer Prev 1991;1:69-74.

45. Hofmann A F, Sjovall J, Kurz G, Radominska A, Schteingart C D, Tint G S, Valacevic Z R, Setchell D R. A proposed nomenclature for bile acids. J Lipid Res 1992;33:599-604.

46. Im E, Martinez J D. Ursodeoxycholic acid (UDCA) can inhibit deoxycholic acid (DCA)-induced apoptosis via modulation of EGFR/Raf-1/ERK signaling in human colon cancer cells. J Nutr 2004;134:483-6.

47. J. Med. Chem. 2002; 44:984.

48. Jass J R, Whitehall V L, Young J, Leggett B A. Emerging concepts in colorectal neoplasia. Gastroenterology 2002;123:862-76.

49. Justman Q A, Clinton G M. Herstatin, an autoinhibitor of the human epidermal growth factor receptor 2 tyrosine kinase, modulates epidermal growth factor signaling pathways resulting in growth arrest. J Biol Chem 2002:277:20618-24.

50. Kawana K, Ikuta T, Kobayashi Y, Gotoh O, Takeda K, Kawajiri K, Molecular mechanism of nuclear translocation of an orphan nuclear receptor, SXR. Mol Pharmacol 2003;63:524-31.

51. Kinzler D W, Vogelstein, B. Lessons from hereditary colorectal cancer. Cell 1996;87:159-70.

52. Klos C, Paumgartner, G., Reichen, J. Cation-anion gap and choleretic properties of rat bile. Am J Physiol 1979;236:E434-E40.

53. Koh T J, Bulitta C J, Fleming J V, Dockray G J, Varro A, Wang T C. Gastrin is a target of the beta-eatenin/TCF-4 growth-signaling pathway in a model of intestinal polyposis. J Clin Invest 2000;106:533-9.

54. Kohler, et al., Eur. J. Immunol. 1976; 6:292.

55. Kohler, et al., Eur. J. Immunol. 1976; 6:511.

56. Kohler, et al., Nature 1975; 256:495.

57. Lee, et al., Nucleic Acids Research 1979; 6:3073.

58. Lichtenstein P, Holm, N. V., Verkasalo, P. K., Iliadou, A., Kaprio, J., Koskenvuo, M., Pukkala, E., Skytthe, A., Hemminki, K. Environmental and heritable factors in the causation of cancer. N Engl J Med 2000;343:78-85.

59. Luttrell L. M. Delia Rocca G J, van Biesen T, Luttrell D K, Lefkowitz R J. Gbetagamma subunits mediate Src-dependent phosphorylation of the epidermal growth factor receptor. A scaffold for G protein-coupled receptor-mediated Ras activation. J Biol Chem 1997;272:4637-44.

60. Martinez J D, Stratagoules E D, LaRue J M, Powell A A, Gause P R, Craven M T, Payne C M, Powell M B, Gerner E W, Earnest D L. Different bile acids exhibit distinct biological effects: the tumor promoter deoxycholic acid induces apoptosis and the ehemopreventive agent ursodeoxycholic acid inhibits cell proliferation. Nutr Cancer 1998;31:111-8.

61. McGettigan P, Henry D. Cardiovascular risk and inhibition of cyclooxygenase: a systematic review of the observational studies of selective and nonselective inhibitors of cyclooxygenase 2. Jama 2006;296:1633-44.

62. McJunkin B, Fromm H, Sarva R P, Amin P. Factors in the mechanism of diarrhea in bile acid malabsorption: fecal pH—a key determinant. Gastroenterology 1981;80:1454-64.

63. Meziane H, Dodart J C, Mathis C, Little S, Clemens J, Paul S M, Ungerer A. Memory-enhancing effects of secreted forms of the beta-amyloid precursor protein in normal and amnestic mice. Proc Natl Acad Sci USA 1998;95:12683-8.

64. Morrison, Science 1985; 229:1202.

65. Nachum Z, Shahal B, Shupak A, Spitzer O, Gonen A, Beiran I, Lavon H, Eynan M, Dachir S, Levy A. Scopolamine bioavailability in combined oral and transdennal delivery. J Pharmacol Exp Ther 2001;296:121-3.

66. Nagengast F M, Grubben, M. J. A. L., van Munster, I. P. Role of bile acids in colorectal carcinogenesis. Eur J Cancer 1995;31A:1067-70.

67. Narisawa T, Fukaura Y, Terada K, Sekiguchi H. Prevention of N-methylnitrosourea-induced colon tumorigenesis by ursodeoxycholic acid in F344 rats. Jpn J Cancer Res 1998;89:1009-13.

68. Narisawa T, Magadia N E, Weisburger J H, Wynder E L. Promoting effect of bile acids on colon carcinogenesis after intrarectal instillation of N-methyl-N′-nitro-N-nitrosoguanidine in rats. J Natl Cancer Instit 1974;53:1093-7.

69. Neuberger, et al., Nature, 1985; 314:268.

70. O'Conner, Neurochem. 1991; 56:560.

71. Ochiai M, Ubagai T, Kawamori T, Imai H, Sugimura T, Nakagama H. High susceptibility of Scid mice to colon carcinogenesis induced by azoxymethane indicates a possible caretaker role for DNA-dependent protein kinase. Carcinogenesis 2001;22:1551-5.

72. Oi, et al., BioTechniques, 1986;4:214.

73. Okano, Neurochem., 1991;56:560.

Oligodeoxynucleotides as Anitsense Inhibitors of Gene Expression, CRC Press, Boca Raton, Fla. 1998.

74. Pai R, Soreghan B, Szabo IL, Pavelka M, Baatar D, Tarnawski AS. Prostaglandin E2 transactivates EGF receptor: a novel mechanism for promoting colon cancer growth and gastrointestinal hypertrophy. Nat Med 2002;8:289-93.

75. Pai R, Tarnawski A S, Tran T. Deoxycholic acid activates beta-catenin signaling pathway and increases colon cell cancer growth and invasiveness. Mol Biol Cell 2004;15:2156-63.

76. Pardi D S, Loftus E V, Jr., Kremers W K, Keach J, Lindor K D. Ursodeoxycholic acid as a chemopreventive agent in patients with ulcerative colitis and primary sclerosing cholangitis. Gastroenterology 2003;124:889-93.

77. Paulsen J E, Steffensen I L, Loberg E M, Husoy T, Namork E, Alexander J. Qualitative and quantitative relationship between dysplastic aberrant crypt foci and tumorigenesis in the Min/+ mouse colon. Cancer Res 2001;61:5010-5.

78. Poley J R, Hofrnann, A. F. Role of fat maldigestion in pathogenesis of steatorrhea in ileal resection. Gastroenterology 1976;71:38-44.

79. Porter J L, Fordtran J S, Santa Ana C A, Emmett M, Hagey L R, Macdonald E A, Hofrnann A F. Accurate enzymatic measurement of fecal bile acids in patients with malabsorption. J Lab Clin Med 2003;141:411-8.

80. Pries J M, Sherman, C. A., Williams, G. C., Hanson, R. F. Hepatic extraction of bile salts in conscious dog. Am J Physiol 1979;236:E191-E7.

81. Qiao D, Chen W, Stratagoules E D, Martinez J D. Bile acid-induced activation of activator protein-1 requires both extracellular signal-regulated kinase and protein kinase C signaling. J Biol Chem 2000;275:15090-8.

82. Qiao D, Gaitonde S V, Qi W, Martinez J D. Deoxycholic acid suppresses p53 by stimulating proteasome-mediated p53 protein degradation. Carcinogenesis 2001;22:957-64.

83. Rang H P, et al, Cholinergic Transmission, Pharmacology, Churchill Livingstone, New York, 1995; Chapter 6.

84. Rasmussen T, Fink-Jensen A. Intravenous scopolamine is potently self-administered in drug-naive mice. Neuropsychopharmacology 2000;22:97-9.

85. Raufman J P, Chen Y, Cheng K, Compadre C, Compadre L, Zimniak P. Selective interaction of bile acids with muscarinic receptors: a case of molecular mimicry. Eur J Pharmacol 2002;457:77-84.

86. Raufman J P, Chen Y, Zimniak P, Cheng K. Deoxycholic acid conjugates are muscarinic cholinergic receptor antagonists. Pharmacology 2002;65:215-21.

87. Raufman J P, Zimniak P, Bartoszko-Malik A. Lithocholyltaurine interacts with cholinergic receptors on dispersed chief cells from guinea pig stomach. Am J Physiol 1998;274:G997-1004.

88. Reddy B S, Narasawa T, Weisburger J H, Wynder E L. Promoting effect of sodium deoxycholate on colon adenocarcinomas in germfree rats. J Natl Cancer Instit 1976;56:441-2.

89. Reddy B S, Wynder E L. Metabolic epidemiology of colon cancer. Fecal bile acids and neutral sterols in colon cancer patients and patients with adenomatous polyps. Cancer 1977;39:2533-9.

90. Renner U D, Oertel R, Kirch W. Pharmacokinetics and pharmacodynamics in clinical use of scopolamine. Ther Drug Monit 2005;27:655-65.

91. Rotin D, Margolis B, Mohammadi M, Daly R J, Daum G, Li N, Fischer E H, Burgess W H, Ullrich A, Schlessinger J. SH2 domains prevent tyrosine dephosphorylation of the EGF receptor: identification of Tyr992 as the high-affinity binding site for SH2 domains of phospholipase C gamma. Embo J 1992;11:559-67.

92. Russo M W, Wei J T, Thiny M T, Gangarosa L M, Brown A, Ringel Y, Shaheen N J, Sandler R S. Digestive and liver diseases statistics, 2004. Gastroenterology 2004;126:1448-53.

93. Sarver, et al. Science 1990;247:1222-1225.

94. Schwartz, et al., Gene Ther. 1996; 3(5):405411.

95. Seger R, Krebs E G. The MAPK signaling cascade. Faseb J 1995;9:726-35.

96. Skehan P, Storeng R, Scudiero D, Monks A, McMahon J, Vistica D, Warren J T, Bokesch H, Kenney S, Boyd M R. New colorimetric cytotoxicity assay for anticancer-drug screening. J Natl Cancer Inst 1990;82:1107-12.

97. Slack B E. The m3 muscarinic acetylcholine receptor is coupled to mitogen-activated protein kinase via protein kinase C and epidermal growth factor receptor kinase, Biochem J 2000;348 Pt 2:381-7.

98. Soler A P, Miller R D, Laughlin K V, Caip N Z, Klurfeld D M, Mullm J M. Increased tight junctional penneability is associated with the development of colon cancer. Carcinogenesis 1999;20:1425-31.

99. Sutherland L A, Bird R P. The effect of chenodeoxycholic acid on the development of aberrant crypt foci in the rat colon. Cancer Lett 1994;76:101-7.

100. Tabata, et al., Cardiovasc. Res. 1997;35(3):470-479.

101. Takahashi M, Nakatsugi S, Sugimura T, Wakabayashi K. Frequent mutations of the beta-catenin gene in mouse colon tumors induced by azoxymethane. Carcinogenesis 2000;21:1117-20.

102. Tsujii M, DuBois R N. Alterations in cellular adhesion and apoptosis in epithelial cells overexpressing prostaglandin endoperoxide synthase 2. Cell 1995;83:493-501.

103. Tsurumi, et al., Circulation 1996;94(12):3281-3290.

104. Ventura A, Kirsch D G, McLaughlin M E, Tuveson D A, Grimm J, Lintault L, Newman J, Reczek E E, Weissleder R, Jacks T. Restoration of p53 function leads to tumour regression in vivo. Nature 2007;445:661-5.

105. Wali R K, Khare S, Tretiakova M, Cohen G, Nguyen L, Hart J, Wang J, Wen M, Ramaswamy A, Joseph L, Sitrin M. Brasitus T A, Bissonette M. Ursodeoxycholic Acid and F6-D3 ihibit aberrant crypt proliferation in the rat azoxymethane model of colon cancer: roles of cyclin D1 and e-cadherin. Can Epid Bio Prev 2002;11:1653-62.

106. Wands, et al, Gastroenterology 1981; 80:225-232.

107. Wang D, Wang H, Guo Y, Ning W, Katkuri S, Wahli W, Desvergne B, Dey S K, DuBois R N. Crosstalk between peroxisome proliferator-activated receptor delta and VEGF stimulates cancer progression. Proc Natl Acad Sci USA 2006;103:19069-74.

108. Wang Q S, Papanilolaou A, Sabourin C L K, Rosenberg D W. Altered expression of cyclin D1 and cyclin-dependent kinase 4 in azoxymethane-induced mouse colon tumorigenesis. Carcinogenesis 1998;19:2001-6.

109. Wess J. Muscarinic acetylcholine receptor knockout mice: novel phenotypes and clinical implications. Annu Rev Pharmacol Toxicol 2004;44:423-50.

110. Winawer S, Fletcher R, Rex D, Bond J, Burt R, Ferrucci J, Ganiats T, Levin T, Woolf S, Johnson D, Kirk L, Litin S, Simmang C. Colorectal cancer screening and surveillance: Clinical guidelines and rationale-Update based on new evidence. Gastroenterology 2003;124:544-60.

111. Wolff, Neuromuscul. Disord. 1997; 7(5):314-318.

112. Xie G, Drachenberg C, Yamada M, Wess J, Raufman J P. Cholinergic agonist-induced pepsinogen secretion from murine gastric chief cells is mediated by M1 and M3 muscarinic receptors. Am J Physiol Gastrointest Liver Physiol 2005;289:G521-9.

113. Xue W, Zender L, Miething C, Dickins R A, Hernando E, Krizhanovsky V, Cordon-Cardo C, Lowe SW. Senescence and tumour clearance is triggered by p53 restoration in murine liver carcinomas. Nature 2007;445:656-60.

114. Yamada M, Miyakawa T, Duitaroy A, Yamanaka A, Moriguchi. T, Makita R, Ogawa M, Chou C J, Xia B, Crawley J N, Felder C C, Deng C X, Wess J. Mice lacking the M3 muscarinic acetylcholine receptor are hypophagic and lean. Nature 2001;410:207-12.

115. Yamada Y. Mori H. Pre-cancerous lesions for colorectal cancers in rodents: a new concept. Carcinogenesis 2003;24:1015-9.

116. Yamada Y, Yoshimi N, Hirose Y, Kawabata K, Matsunaga K, Shimizu M, Hara A, Mori H. Frequent beta-catenin gene mutations and accumulations of the protein in the putative preneoplastic lesions lacking macroscopic aberrant crypt foci appearance, in rat colon carcinogenesis. Cancer Res 2000;60:3323-7.

117. Yang W L, Frucht H, Cholinergic receptor up-regulates COX-2 expression and prostaglandin E(2) production in colon cancer cells. Carcinogenesis 2000;21:1789-93.

What is claimed is:

1. A method for reducing proliferation of colon tumor ceils, the method comprising administering to the cells an effective amount of at least one agent that reduce M3 muscarinic receptor-mediated transactivation of at least one epidermal growth factor receptor.

2. The method of claim 1, wherein the at least one agent comprises an M3 muscarinic receptor antagonist, anti-M3 muscarinic receptor antibody, anti-M3 muscarinic receptor nucleic acid sequence, ribozymes, or a combination thereof.

3. The method of claim 1, wherein the anti-M3 muscarinic receptor nucleic acid sequence is a double-stranded RNA, an antisense oligonucleotide, or a combination thereof.

4. The method of claim 3, wherein the double-stranded RNA is a small interfering RNA.

5. The method of claim 1, wherein the colon tumor cells are human cells.

6. A method for inhibiting proliferation of colon tumor cells, the method comprising administering to a subject in need of treatment an effective amount of at least one agent that inhibits the expression of M3 muscarinic receptor.

7. The method of claim 6, wherein the at least one agent comprises anti-M3 muscarinic receptor antibody, anti-M3 muscarinic receptor nucleic acid sequence, ribozymes, or a combination thereof.

8. The method of claim 7, wherein the anti-muscarinic receptor nucleic acid sequence is a double-stranded RNA, antisense oligonucleotide, or combination thereof.

9. The method of claim 8, wherein the double-stranded RNA is a small interfering RNA.

10. The method of claim 6, wherein the colon tumor cells are human cells.

11. A method for inhibiting tumor metastasis in a subject, the method comprising administering to a subject in need of treatment a therapeutically effective amount of at least one agent that reduces M3 muscarinic receptor-mediated transactivation of at least one epidermal growth factor receptor, or inhibits M3 receptor expression.

12. The method of claim 11, wherein the at least one agent comprises M3 muscarinic receptor antagonist, anti-M3 muscarinic receptor antibody, anti-M3 muscarinic receptor nucleic acid sequence, ribozymes, or a combination thereof.

13. The method of claim 12, wherein the anti-muscarinic receptor nucleic acid sequence is a double-stranded RNA, antisense oligonucleotide, or a combination thereof.

14. The method of claim 13, wherein the double-stranded RNA is a small interfering RNA.

15. The method of claim 11, wherein the subject is a human.

16. The method of claim 15, wherein the human is genetically pre-disposed to tumor development.

17. The method of claim 16, wherein the subject has Familial Adenomatous Polyposis or a phenotypically similar disorder.

18. A method of reducing the likelihood of the development of a colon tumor in a subject, the method comprising administering to the subject a therapeutically effective amount of at least one agent to reduce M3 muscarinic receptor-mediated transactivation of at least one epidermal growth factor receptor.

19. The method of claim 18, wherein the at least one agent comprises M3 muscarinic receptor antagonist, anti-M3 muscarinic receptor antibody, double-stranded RNA, antisense oligonucleotides, ribozymes, or a combination thereof.

20. The method of claim 19, wherein the anti-muscarinic receptor antibody is an anti-muscarinic M3 receptor antibody.

21. The method of claim 19, wherein the double-stranded RNA is a small interfering RNA.

22. The method of claim 21, wherein the subject is a human.

23. The method of claim 22, wherein the human is genetically pre-disposed to tumor development.

24. The method of claim 23, wherein subject has Familial Adenomatous Polyposis.

25. A transgenic animal, said animal comprising a genome encodes at least two transgenes, said transgenes comprising a mutated murine M₃ muscarinic receptor gene and a mutated adenomatous polyposis coli gene, wherein the mutated murine muscarinic M₃ receptor gene results in a reduction in M₃ muscarinic receptor gene expression and at least an about a 50% reduction in tumor proliferation in the colon compared to a wild-type mouse.

26. The transgenic mouse of claim 25, wherein the mutated human M₃ receptor gene results in at least an about 80% reduction in tumor proliferation in the colon compared to a wild-type mouse.

27. The transgenic mouse of claim 26, wherein the mouse exhibits reduced tumor proliferation in the colon upon administration of a tumorogenic agent when compared to a wild-type mouse.

28. The method of claim 27, wherein the tumorogenic agent is a carcinogen.
